(19) United States
(12) Patent Application Publication
(10) Pub. No.: US 2025/0144270 A1
(43) Pub. Date: May 8, 2025

(54) PROTEIN-BASED SURFACE COATINGS

(71) Applicant: THE GOVERNORS OF THE UNIVERSITY OF ALBERTA, Edmonton (CA)

(72) Inventors: Hongbo Zeng, Edmonton (CA); Ziqian Zhao, Edmonton (CA)

(21) Appl. No.: 18/941,432

(22) Filed: Nov. 8, 2024

Related U.S. Application Data
(60) Provisional application No. 63/597,207, filed on Nov. 8, 2023.

(51) Int. Cl. A61L 27/34; A61L 27/54

(52) U.S. Cl. CPC A61L 27/34; A61L 27/54; A61L 2300/252; A61L 2420/00

(57) ABSTRACT

Embodiments herein generally relate to protein-based surface coatings. In some examples, disclosed embodiments relate to a process for synthesizing a protein-based surface coating, comprising conjugating acrylate or (meth-)acrylate monomer to serum albumin molecules. In at least one example, the conjugation step comprises: reducing native serum albumin molecules to produce free thiol groups; reacting the reduced serum albumin with (meth-)acrylate or acrylate monomers to react carbon-carbon double bonds with the free thiol groups; and inducing (meth-)acrylate or acrylate polymerization.

### FIELD

The present invention generally relates to surface coatings, and more particularly, to protein-based surface coatings.

### BACKGROUND

Implantable medical devices (IMDs), such as implantable sensors for chronic disease diagnosis, joint replacements such as knee replacements, and artificial blood vessels, are well known and medically useful devices. IMDs, however, can suffer from dysfunction and infections due to irreversible biofouling. In particular, foulants (e.g., microorganisms, biomolecules such as proteins, and/or metabolites from surrounding biofluids) often irreversibly adhere to the working surfaces of IMDs during their long-lasting operation, and incrementally impair the electrochemical and biological properties of the IMDs. In turn, this causes unpredictable device dysfunction and serious infections.

### SUMMARY

In one aspect, disclosed are protein-polymer conjugates comprising acrylate or methacrylate polymers conjugated to protein molecules, which may preferably comprise serum albumin proteins, such as bovine serum albumin.

In some embodiments, the polymer comprises a methacrylate polymer comprising zwitterionic methacrylate such as poly sulfobetaine methacrylate (SMBA).

In some embodiments, the polymer comprises a methacrylate polymer comprising pH-responsive amino groups, such as poly 2-Aminoethyl methacrylate (PAMA).

In some embodiments, the polymer comprises a methacrylate polymer comprising a quaternary ammonium groups, such as poly 2-(methacryloyloxy)ethyltrimethylammonium chloride (PMTAC), poly 2-Methacryloyloxyethyl phosphorylcholine (PMPC), poly 3-Sulfopropyl methacrylate potassium salt (PSPAK), poly 2-Hydroxyethyl methacrylate (PHEMA), or poly dopamine methacrylamide (PDMA).

In some embodiments, the polymer comprises an acrylate polymer comprising poly Sodium acrylate (PSA) monomers.

In another aspect, disclosed is a method of coating a substrate comprising step of contacting the substrate with a polymer-protein conjugate described herein, in an aqueous solution, such that the conjugate binds non-covalently with the substrate. In some embodiments, the aqueous solution has a salt concentration and/or a pH level which has been varied to increase the binding of the conjugate to the substrate.

In another aspect, disclosed is a method of conjugating acrylate or methacrylate monomers having carbon-carbon double bonds to proteins, comprising the steps of:
reducing the proteins to produce free thiol groups;
contacting the reduced proteins with the methacrylate or acrylate monomers to react the carbon-carbon double bonds to the free thiol groups; and
polymerizing the methacrylate or acrylate monomers.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRA WINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

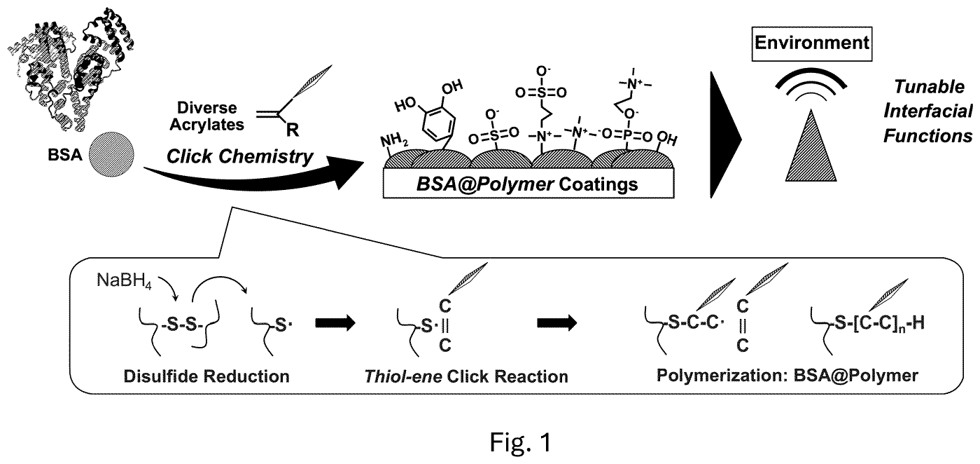

FIG. 1 is a schematic showing the process of synthesizing BSA@Polymer, with diverse functional groups, via thiol-ene click chemistry.

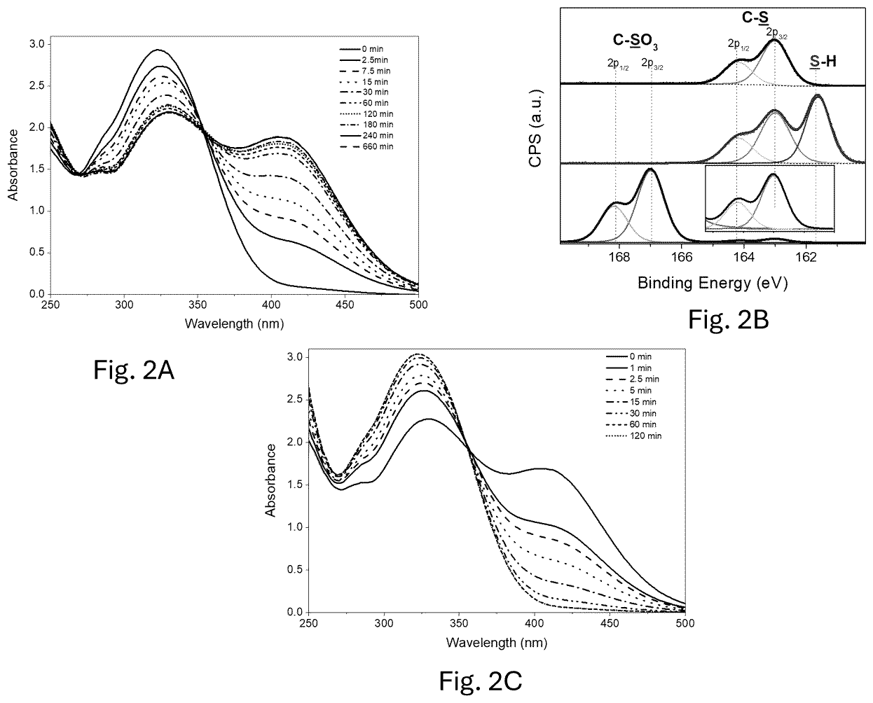

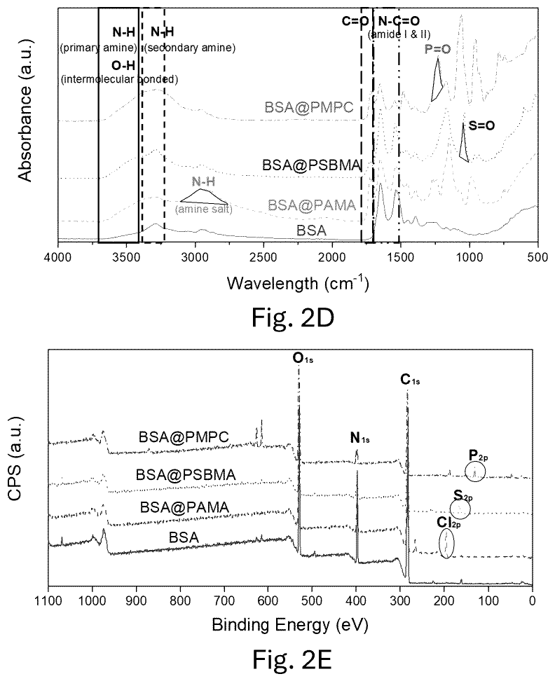

FIGS. 2A-2C show various plots relating to synthesis of engineered protein BSA@Polymer, with diverse functional groups, via click chemistry and including: (i) UV-Vis spectra of Ellman's reagent to detect the free thiol groups in protein solution during the reduction by NaBH₄ (FIG. 2A); (ii) XPS high-resolution S deconvolved spectra of BSA, re-BSA, and BSA@PSBMA (FIG. 2B); (iii) VU-Vis spectra of Ellman's reagent to detect the free thiol groups during reBSA reacted with SBMA monomers (FIG. 2C); (iv) FTIR spectra (FIG. 2D); and (v) XPS survey scan of several typical synthesized BSA@Polymers (FIG. 2E).

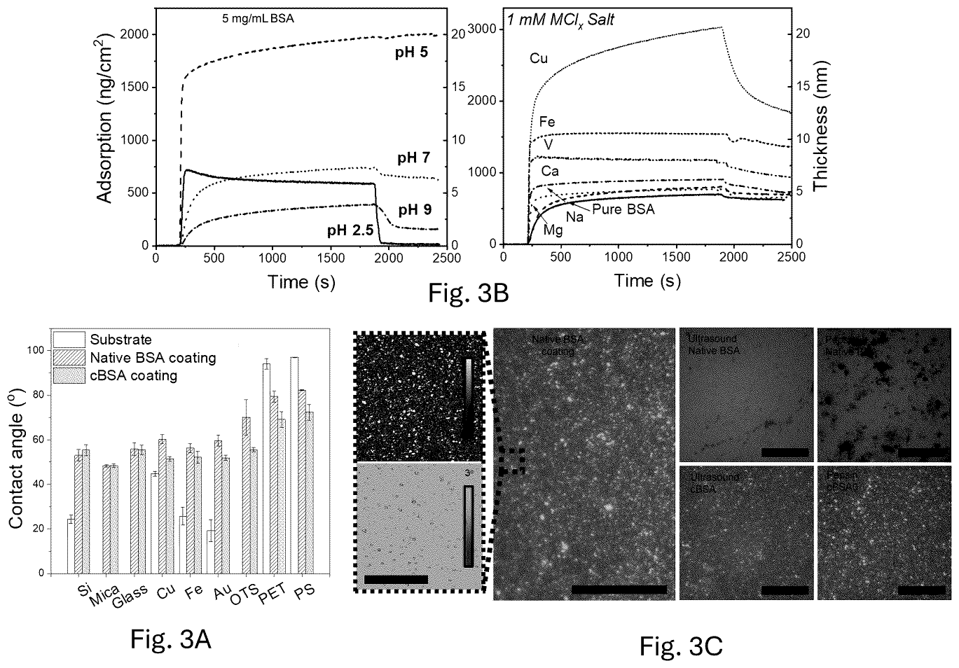

FIGS. 3A-3C show various plots and images relating to the concept of a bioinspired engineered protein for universal anchoring and versatile surface functions, including: (i) static water contact angle on bare substrates and substrates coated with native BSA protein and the crosslinked BSA protein (cBSA) (FIG. 3A), wherein the values represent the mean and the standard deviation (n≥3); (ii) tuning the coating thickness via changing solution pH value and salinity, measured by QCM-D (FIG. 3B); and (iii) protein coatings treated in harsh conditions (FIG. 3C), wherein scale bars are 40 μm.

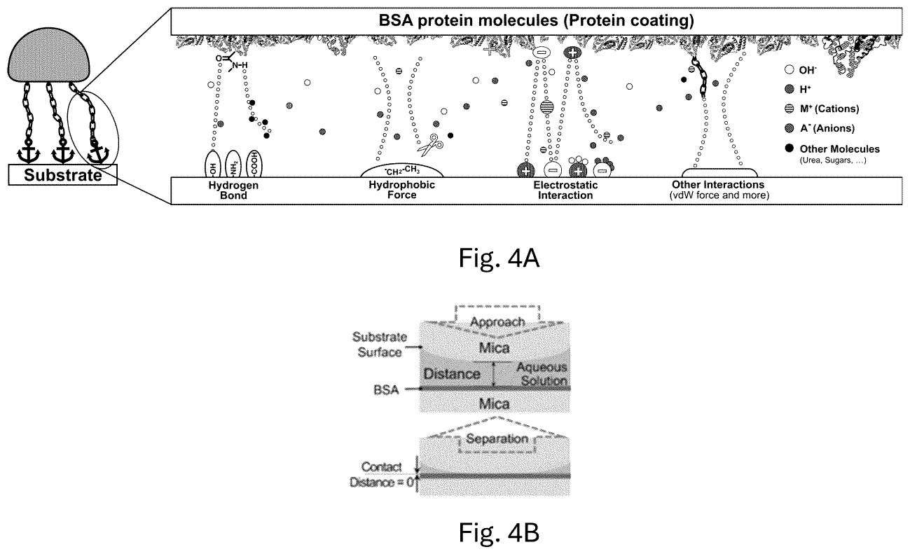

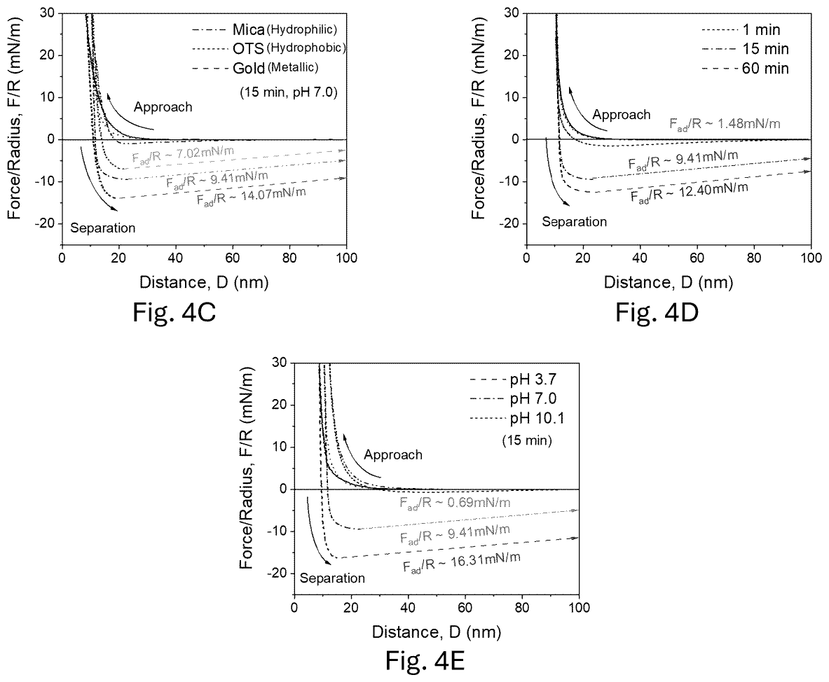

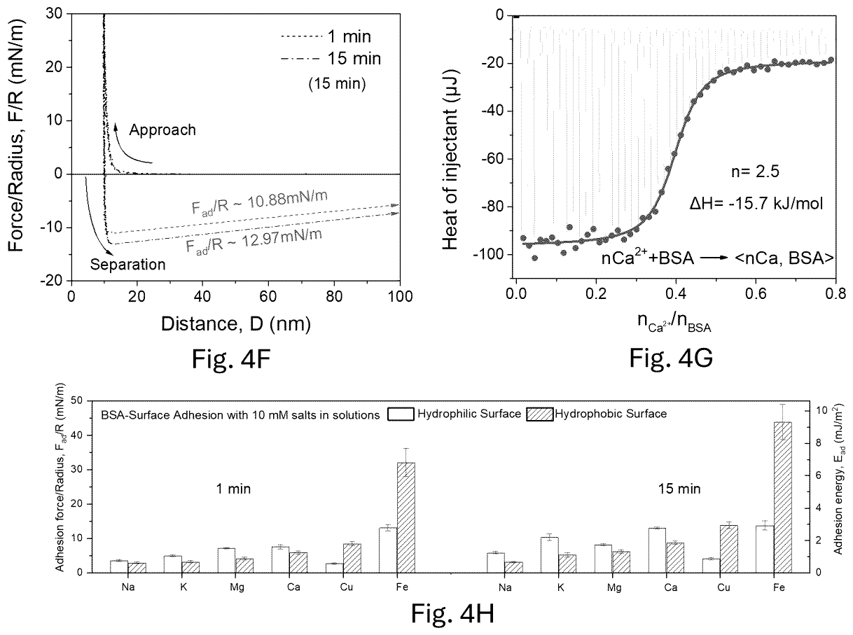

FIGS. 4A-4H show various plots and images relating to molecular interactions between protein coatings and substrates, including: (i) a schematic showing major interfacial molecular interactions enabling BSA anchoring (FIG. 4A); (ii) typical experimental configurations in SFA force measurements (FIG. 4B); (iii) interfacial interaction force profiles (normalized force-distance, F/R-D) of protein coating to different substrate surfaces (FIG. 4C), and to mica surfaces with different contact time (FIG. 4D) in 1 mM NaCl solutions at various pH (FIG. 4E); (iv) interfacial interaction force profiles between protein-coating and mica surface in 10 mM CaCl₂) solution (FIG. 4F); (v) ITC test between Ca²⁺ ions and BSA protein (FIG. 4G); and (vi) Normalized adhesion force (Fad/R) and adhesion energy (E_{ad}) of protein coatings and mica (hydrophilic surface) or OTS (hydrophobic surface) determined in aqueous solutions with the addition of various salts (10 mM NaCl, KCl, MgCl₂, CaCl₂), CuCl₂, and FeCl₃) (FIG. 4H) Values represent the mean and the standard deviation (n≥3).

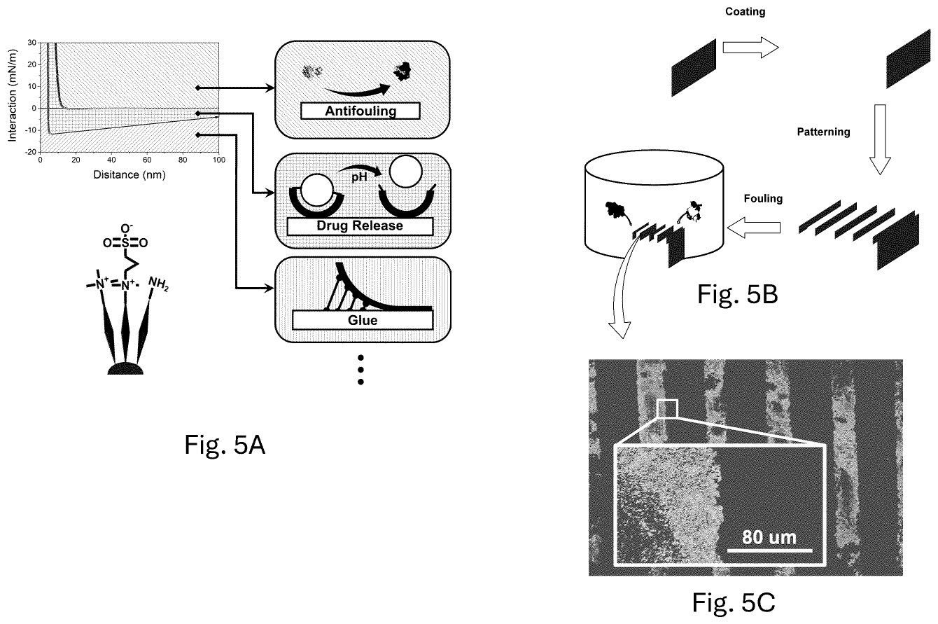

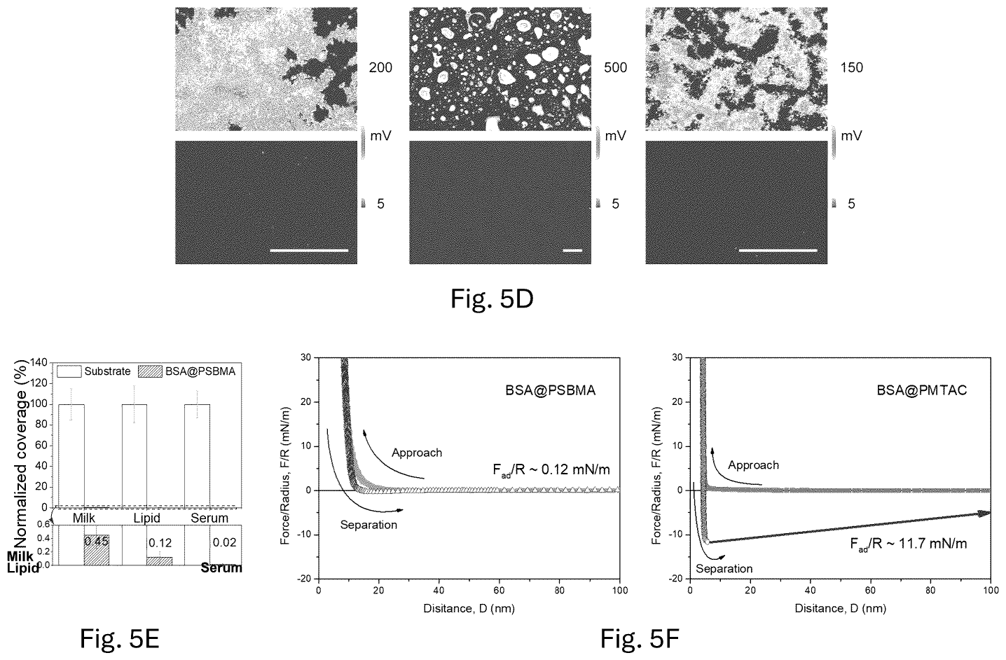

FIGS. 5A-5F show various plots and images relating to multifunctional BSA@Polymer coatings and BSA@PSBMA antifouling coating, including: (i) interfacial interactions of different BSA@Polymer coatings to substances from the surrounding environment and potential applications (FIG. 5A); (ii) preparation of BSA@PSBMA antifouling coating, fouling test (FIG. 5B), and corresponding foulants distribution mapped by O-PTIR (FIG. 5C); (iii) O-PTIR mappings of foulants on the bare substrate surfaces and BSA@PSBMA coated surfaces (FIG. 5D), and corresponding fouling coverage in milk, lipid, and bovine serum solutions (FIG. 5E); (iv) interfacial interactions (normalized force-distance, F/R-D, profiles) between mica surfaces and BSA@PSBMA/BSA@PMTAC. Scale bars in (FIG. 5D) are 500 μm, and values in (FIG. 5E) represent the mean and the standard deviation (n≥3).

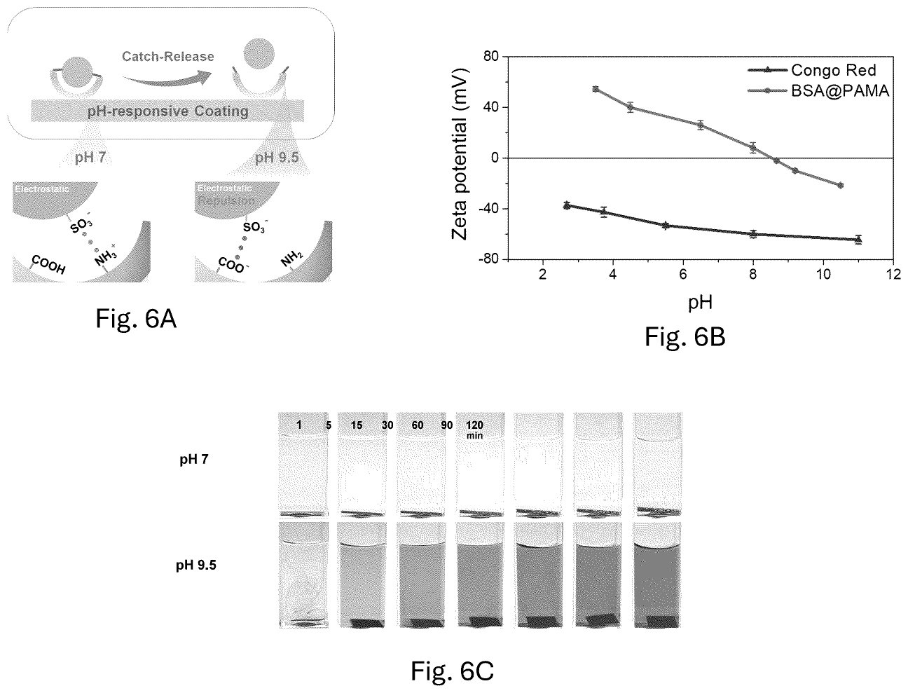

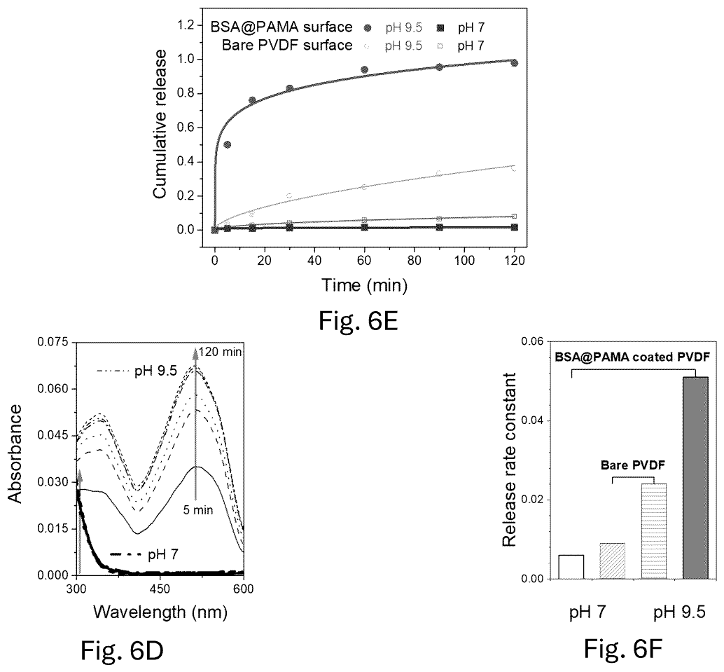

FIGS. 6A-6F show various plots and images relating to BSA@Polymer: (i) interfacial interactions of BSA@PAMA coatings with model drug (Congo Red) at different pH (FIG. 6A); (ii) Zeta potential of BSA@PAMA and congo red (FIG. 6B); (iii) optical image: BSA@PAMA-coated PVDF membranes release drugs at different time (FIG. 6C); (iv) UV-Vis spectra of the solutions with the released drug in FIG. 5c (FIG. 6D); (v) cumulative release of model drug Congo red of different surfaces (FIG. 6E) and their release rate constant (FIG. 6F).

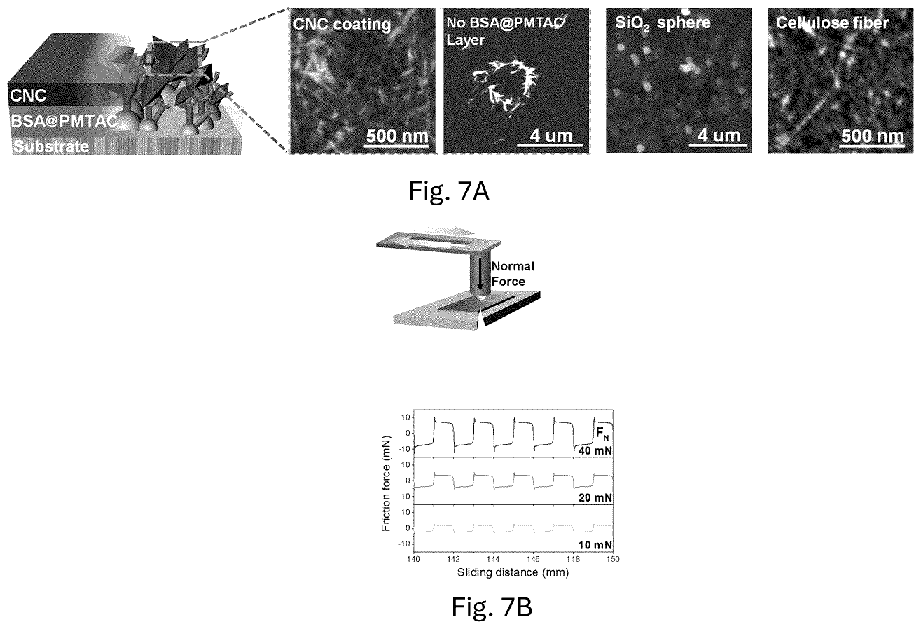

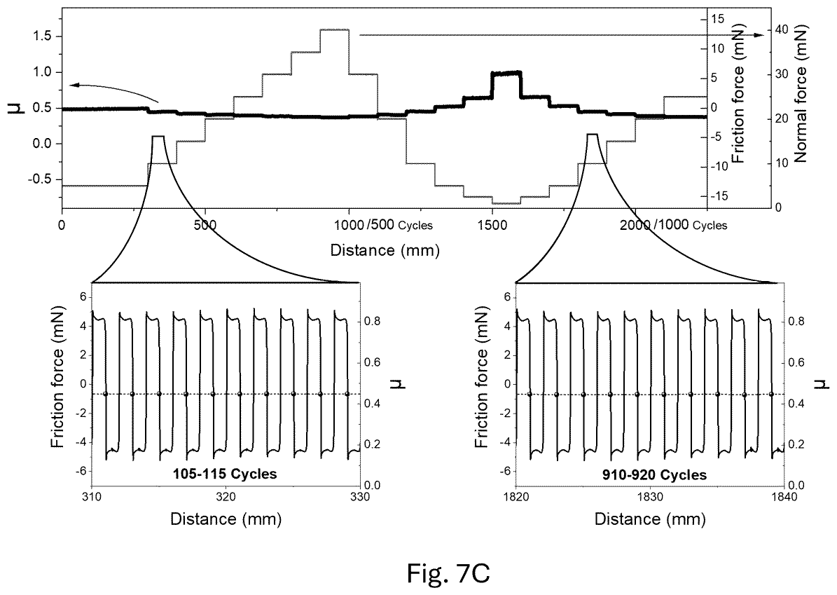

FIGS. 7A-7C shows various images and plots relating to BSA@PMTAC adhesive coatings as the interfacial binder, including: (i) CNC, silica sphere, and cellulose fiber integrated on the BSA@PMTAC coating (FIG. 7A); (ii) the experimental setup of nanotribometer for reciprocating ball-on-disk friction test and typical friction forces with the normal loading force of 10, 20, and 40 mN (FIG. 7B); and (iii) Friction coefficient change under different loading forces during >1000 friction cycles (FIG. 7C).

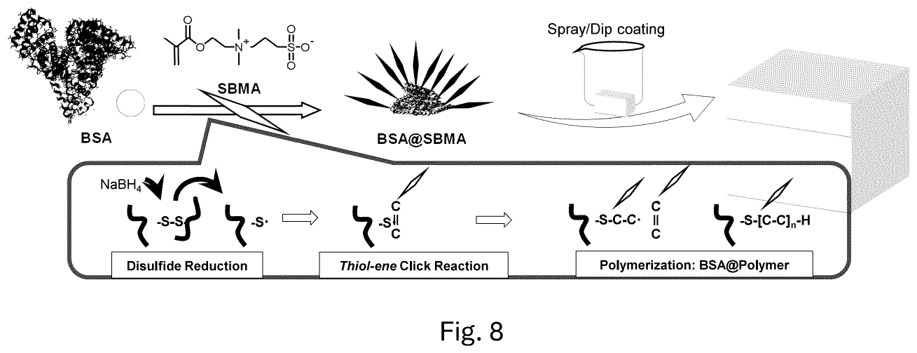

FIG. 8 is a schematic diagram showing the process of synthesizing BSA@PSBMA via thiol-ene click chemistry.

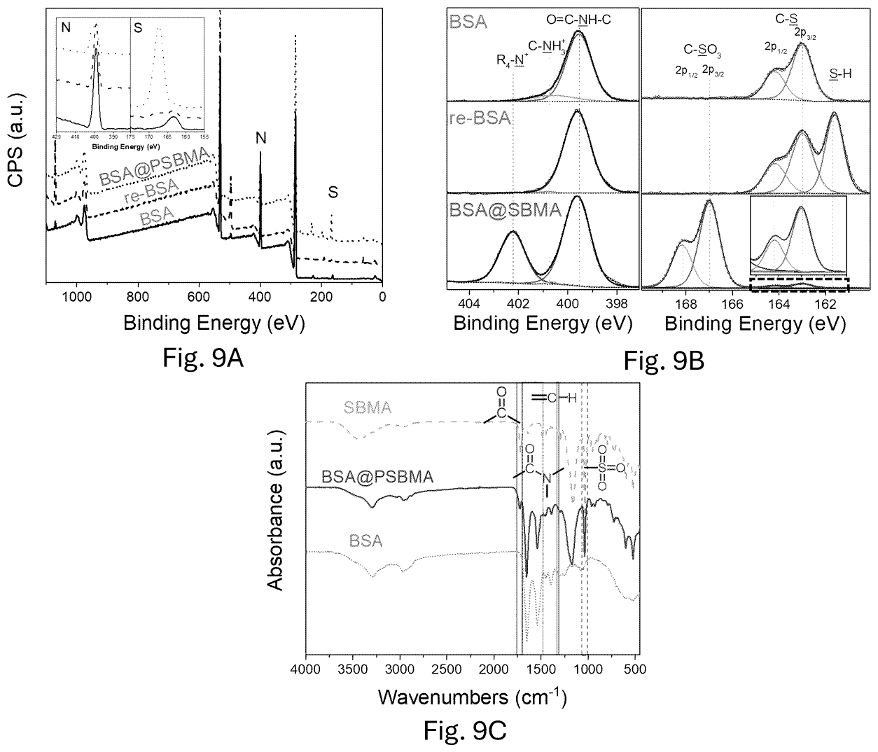

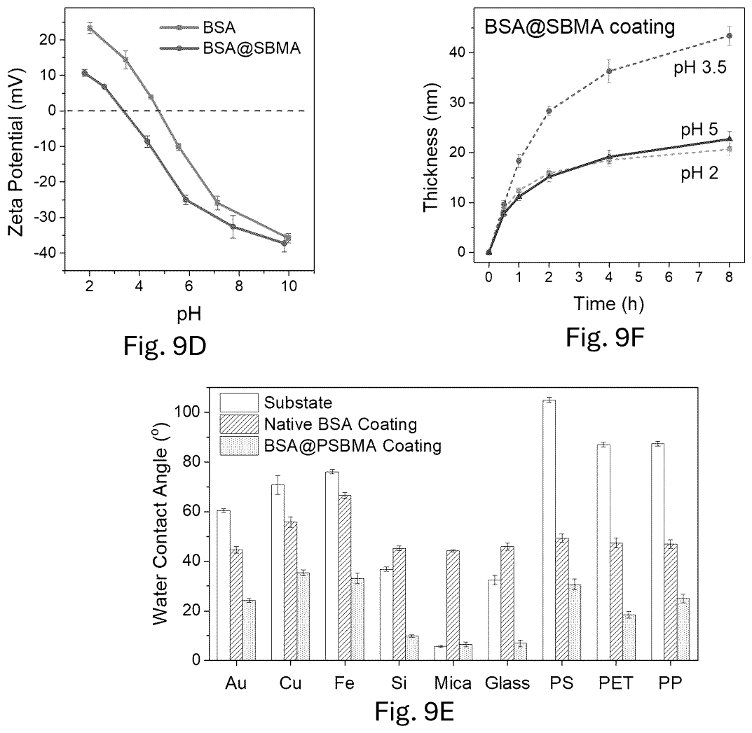

FIGS. 9A-9E are various plots and images relating to the preparation of the engineered protein coating, and showing: (i) XPS wide scan spectra of BSA, re-BSA, and BSA@PSBMA (FIG. 9A), and corresponding high-resolution N and S deconvolution spectra (FIG. 9B); (ii) FTIR spectra of BSA, SBMA, and BSA@PSBMA (FIG. 9C); (iii) zeta potential (FIG. 9D); (iv) static water contact angle on various surfaces (FIG. 9E); and (v) thickness change of BSA@PSBMA coating over coating time under different pH conditions measured using a surface forces apparatus (SFA) (FIG. 9F). Values in (FIGS. 9D-9F) represent the mean and the standard deviation (n=3).

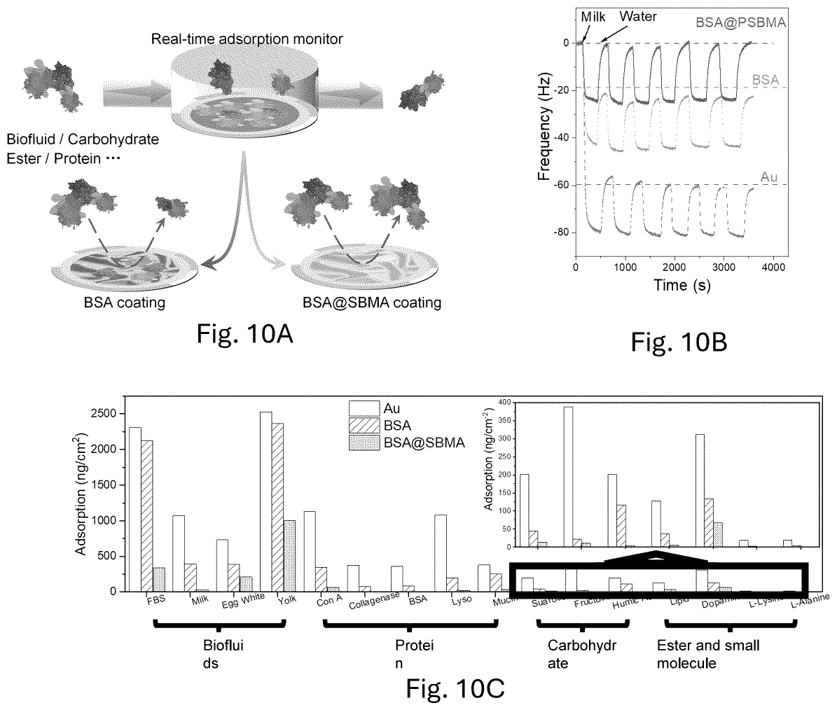

FIGS. 10A-10C show various plots and images relating dynamic adsorption of biofoulants on QCM-D sensors, including: (i) a schematic diagram illustrating dynamic adsorption of foulants on the bare Au sensors and Au sensors coated by native BSA and BSA@PSBMA sensors (FIG. 10A); (ii) change in resonance frequency associated with the adsorption of milk on different sensors during the alternative rinsing with milk and water (FIG. 10B); and (iii) adsorption of biofluids, proteins, carbohydrates, and small molecules on different sensors after rinsing with water (FIG. 10C).

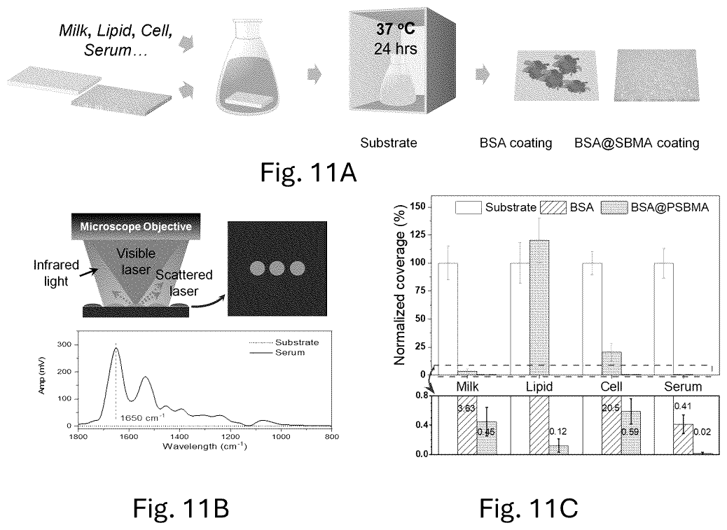

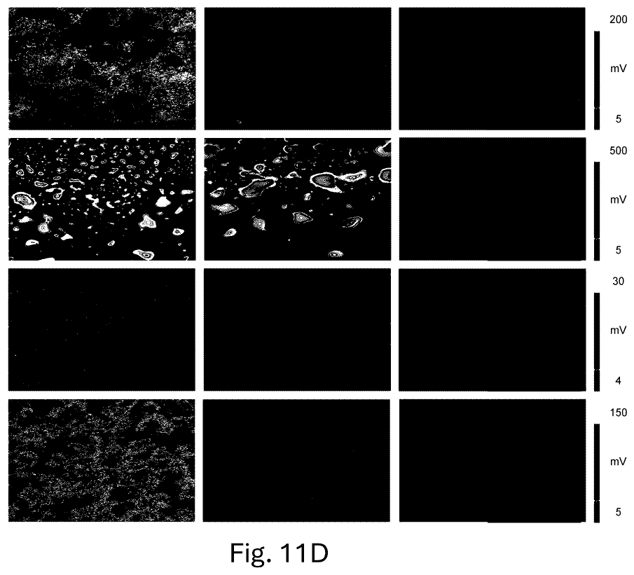

FIGS. 11A-11D show coating antifouling performance in bulk fouling tests, including: (i) a diagram illustrating the processes of macroscale bulk tests (FIG. 11A); (ii) working principle of O-PTIR and O-PTIR spectra of Au substrate and foulants in milk (FIG. 11B); (iii) O-PTIR mappings of foulants on the bare substrate surfaces and surfaces coated by native BSA and BSA@PSBMA (FIG. 11C); and (iv) corresponding fouling coverage in milk, lipid, CHO cells, and bovine serum solutions (FIG. 11D). Values in FIG. 11D represent the mean and the standard deviation (n=6).

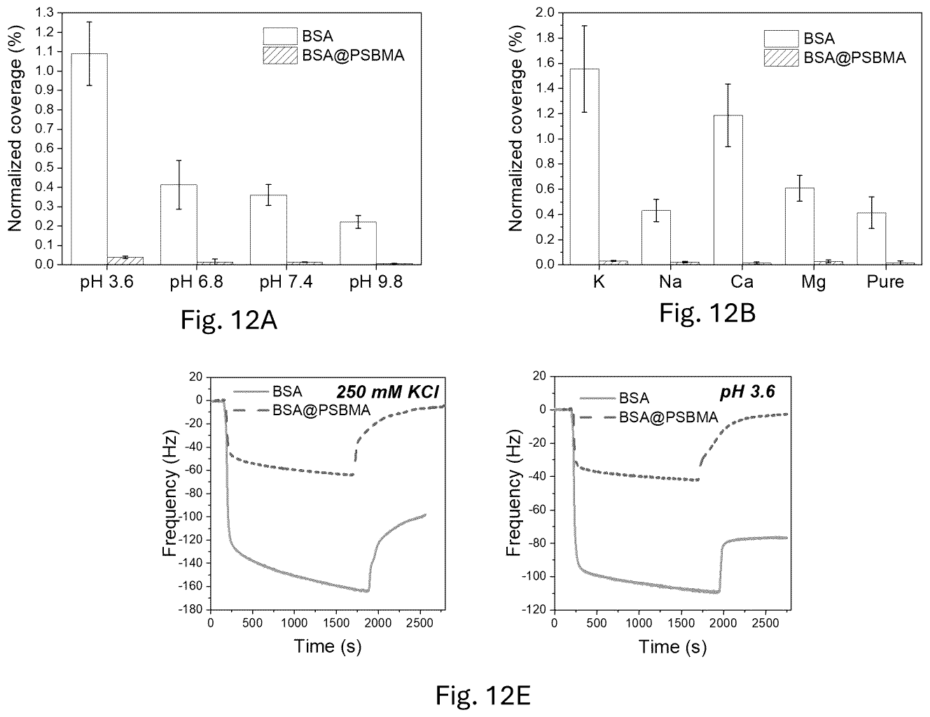

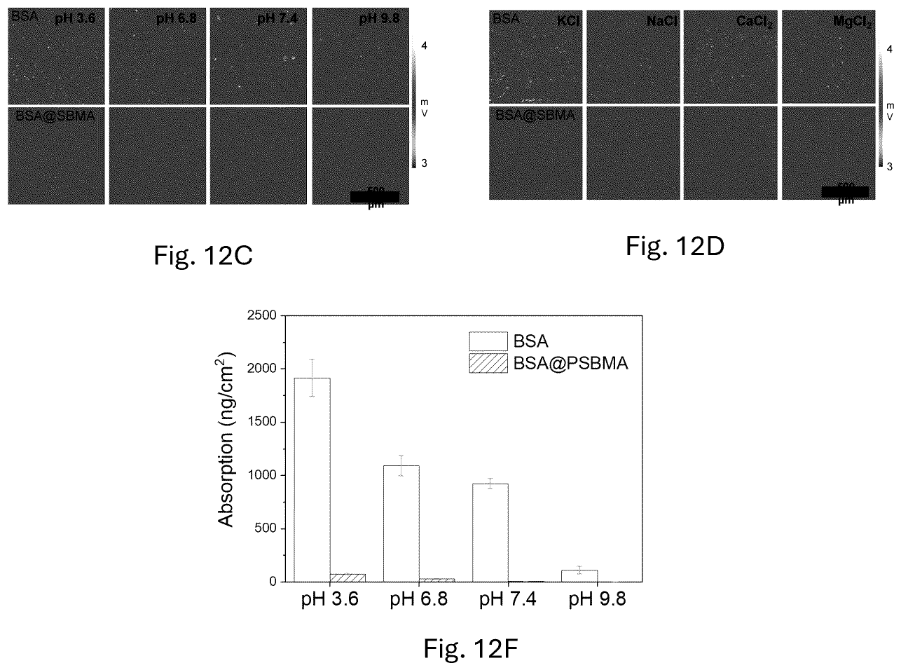

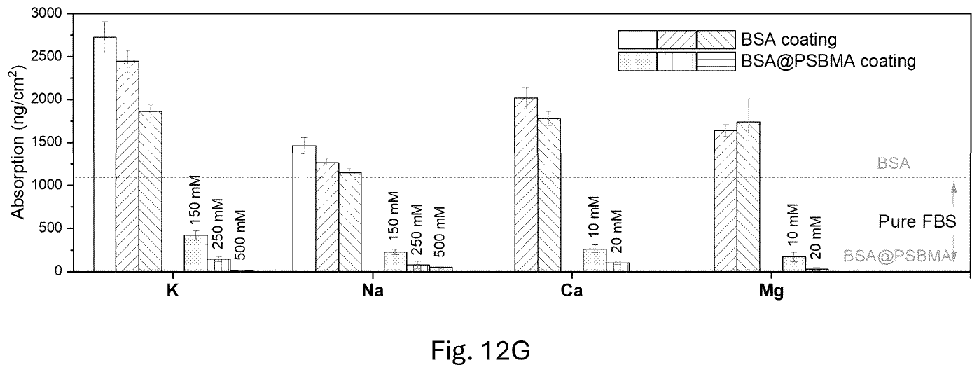

FIGS. 12A-12G shows various plots and images for coating antifouling performance in environments with diverse pH and salinity, and including: (i) normalized coverage of biofoulants on native BSA-coated and BSA@PSBMA-coated surfaces and corresponding O-PTIR mappings incubated in fetal bovine serum (FBS) with diverse pH (FIGS. 12A, 12C) and salinity (FIGS. 12B, 12D); and (ii) Dynamic adsorption of FBS with 250×10−3 m KCl or pH 3.6 on BSA-coated and BSA@PSBMA-coated sensors FIG. 12E), and corresponding adsorption amount of FBS with diverse pH (FIG. 12F) and salinity (FIG. 12G), measured by QCM-D. (Dash line indicates the amount of adsorption of pure FBS on different surfaces). Values represent the mean and the standard deviation (n=3).

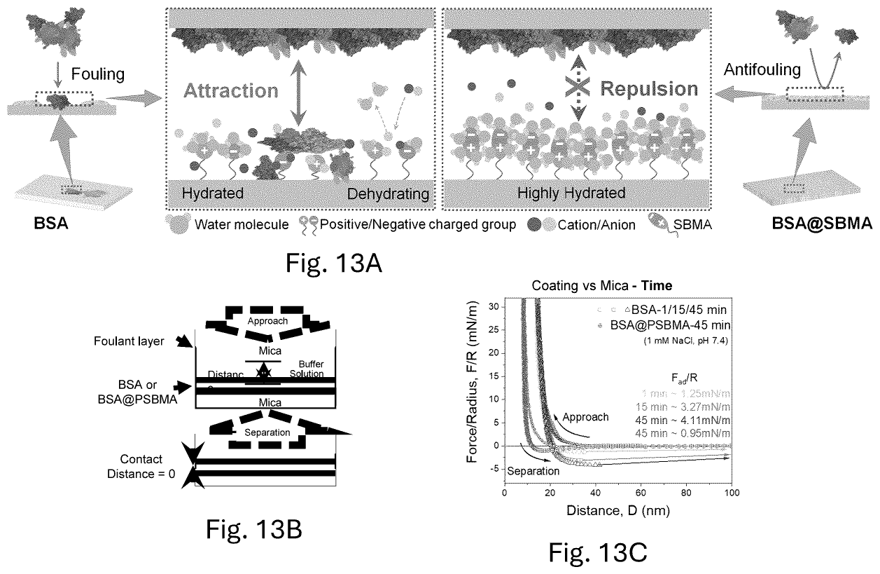

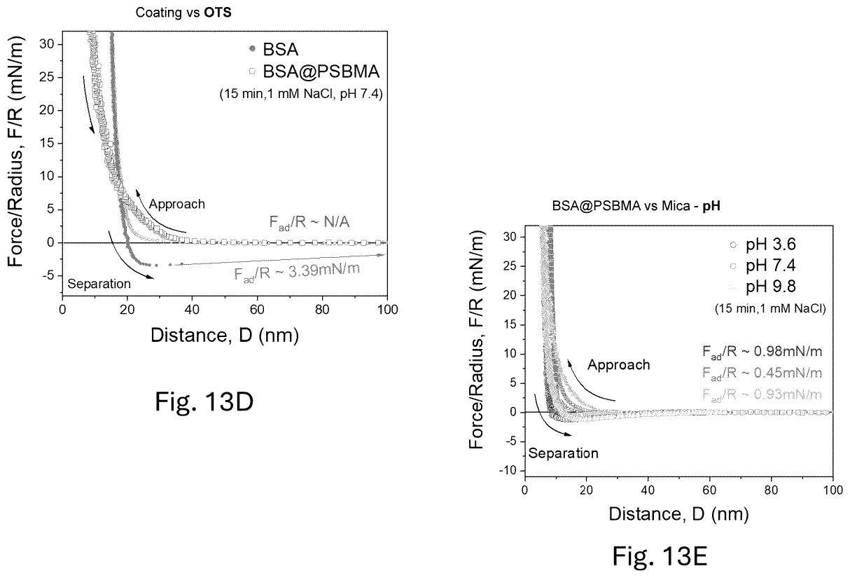

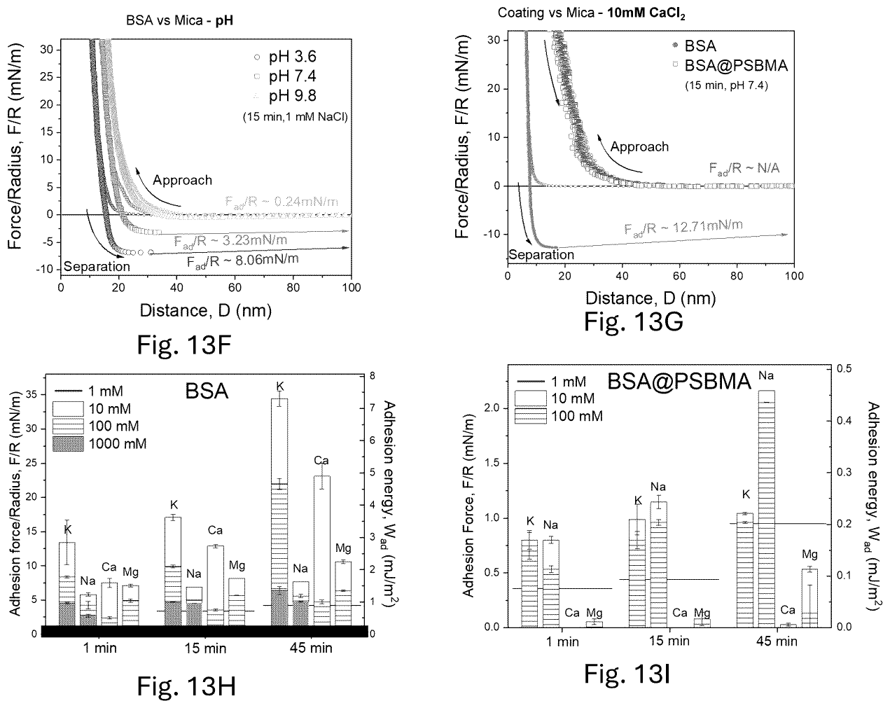

FIGS. 13A-13I show various plots and images for interfacial interactions between foulants and coating surfaces measured by SFA force measurements, including: (i) a schematic diagram showing fouling antifouling phenomena (FIG. 13A); (ii) experimental configurations in SFA force measurements (FIG. 13B). Interfacial interactions (normalized force-distance, F/R-D, profiles) between protein-coated surfaces; (iii) hydrophilic mica surfaces (FIG. 13C), or hydrophobic OTS surfaces (FIG. 13D); (iv) interactions between mica surfaces and BSA@ PSBMA-coated (FIG. 13E), or native BSA-coated surfaces in solutions with different pH. (FIG. 13F); (v) interactions between protein-coated surfaces and mica surfaces in a solution with 10×10−3 m CaCl2 FIG. 13G); and (vi) adhesion force and energy between mica surfaces and native BSA-coated (FIG. 13H) or BSA@PSBMA-coated surfaces (FIG. 13I) in solutions with various salinity at different contact time. Values in FIGS. 13H and 13I represent the mean and the standard deviation (n=3).

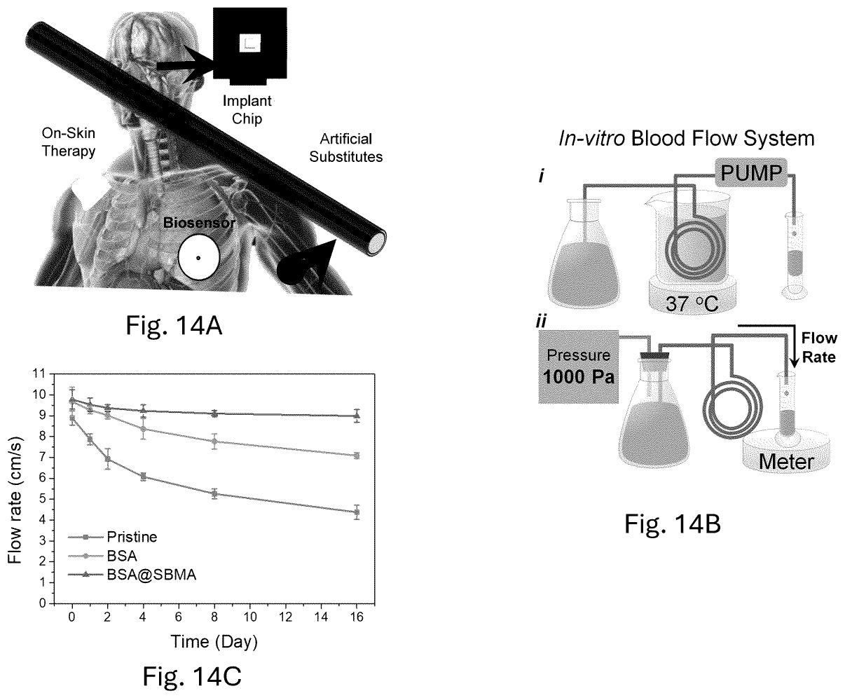

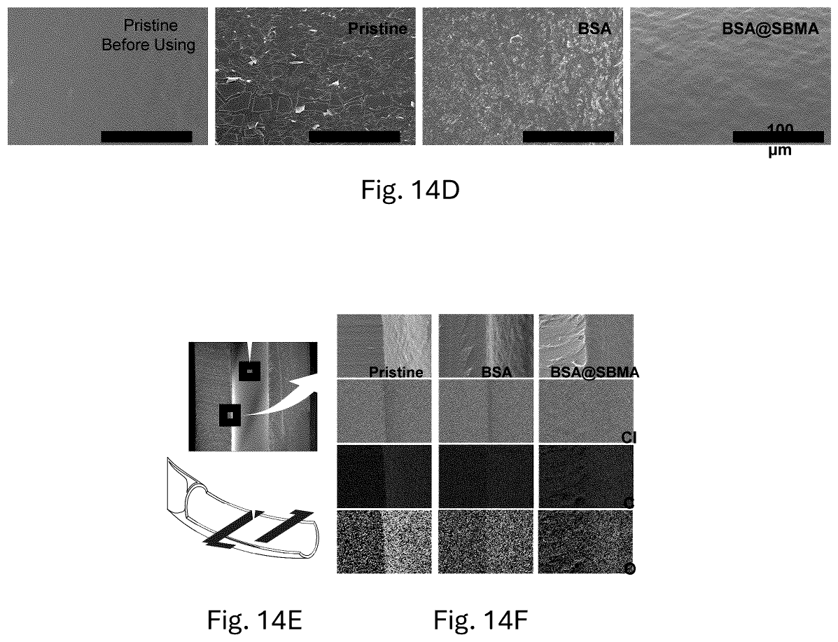

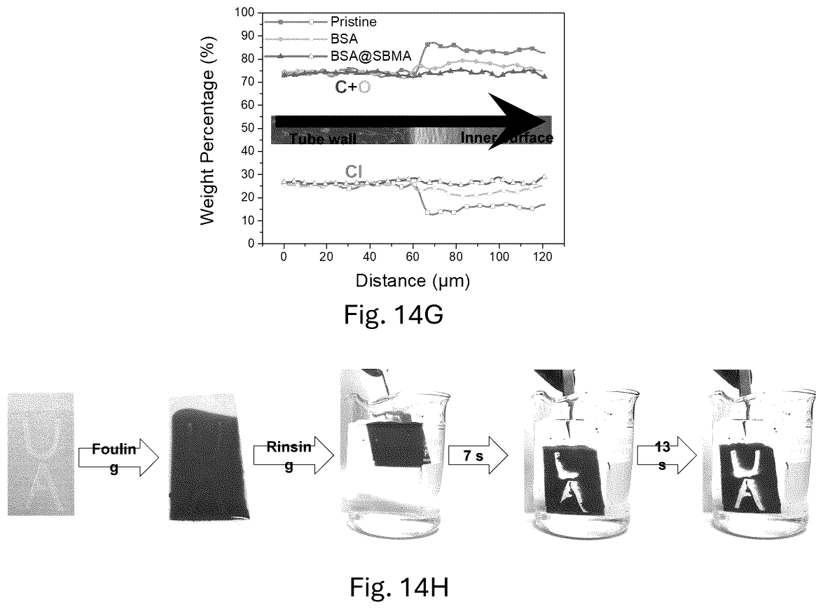

FIGS. 14A-14H show demonstrations of using BSA@PSBMA coating for biofouling resistance in artificial blood vessels, including: (i) schematic of antifouling applications for biomedical needs (FIG. 14A); (ii) in vitro blood flow system (FIG. 14B); (iii) blood flowing rate for pristine, BSA-coated, and BSA@PSBMA-coated medical tubes over operation time (FIG. 14C); (iv) SEM images: the morphology for the inner surfaces of different tubes after 16 d of blood flowing (FIG. 14D); (v) intersection of a medical tube (FIG. 14E); (vi) EDS mapping (FIG. 14F); and (vii) linescan showing the compositions change from the tube wall to the inner surface of different medical tubes (FIG. 14G); and (vii) self-cleaning tests for a BSA@PSBMA coating surface with “UA” pattern (FIG. 14H). Values in (FIG. 14C) represent the mean and the standard deviation (n=3).

### DETAILED DESCRIPTION OF THE EMBODIMENTS

### I. Definitions

Any term or expression not expressly defined herein shall have its commonly accepted definition understood by a person skilled in the art. As used herein, the following terms have the following meanings.

“Antifouling” refers to the ability of a material to remove or prevent biofouling.

“Biofouling” refers to the accumulation of microorganisms, biomolecules such as proteins and/or metabolites on a device surface, which may impair the function of the device.

“Albumin” is a family of globular proteins, the most common of which are “Serum albumins” which are circulating proteins found in plasma. Bovine serum albumin (BSA) is derived from cows. A full length BSA precursor polypeptide is 607 amino acids in length, which is cleaved to form a BSA protein that contains 583 amino acids in length. As used herein, “BSA” refers to the full native BSA molecule, as well as precursors, derivatives or engineered versions which substantially retain BSA functionality of binding non-specifically to a substrate.

“Polyacrylate” and “polymethacrylate” include synthetic polymers produced by polymerization of acrylic and methacrylic esters. Preferably, the polymers have a chain length of greater than about 20 monomer units, and more preferably in the range of about 50 to about 100 monomer units.

This description uses [protein]@ [polymer] nomenclature to describe the conjugates disclosed herein. For example, BSA@PSBMA denotes a conjugate comprising bovine serum albumin and polysulfobetaine methacrylate.

### II. General Overview of Coatings

Embodiments herein generally relate to protein-based surface coatings, comprising a polymer-protein conjugate, which may have different useful functions, such as an antifouling coating, or a pH-responsive drug carrier coating, or a nanomaterial interfacial binding coating. In some examples, the coatings can be applied in biomedical applications in complex biological conditions.

In at least one aspect, disclosed embodiments comprise medical devices or substrates coated with engineered protein-polymer conjugate, where a polymer such as a polyacrylate or a polymethacrylate are grafted on a protein molecule which binds non-specifically to the substrate surface. The disclosed conjugates are inspired by the ‘root-leaf’ structure of grass. More generally, the protein portion of the conjugate serves as a ‘root’ for surface-independent anchoring on target substrates, via various molecular interactions. For example, the protein portion facilitates the surface-independent coating of engineered protein on various substrates (e.g., metallic, organic, and inorganic substrates), achieving a robust and compact coating, even under harsh conditions, including ultrasound, varying pH, surfactants attacks, and enzymatic digestion treatment. The protein portion of the conjugate may be bound to a surface through a simple dipping/spraying method.

Many proteins can be used in different embodiments, such as globular proteins such as albumin, hemoglobin, lysozyme, which are examples of proteins displaying surface-independent anchoring or binding, without covalent bonding. Preferred proteins are soluble in water and have accessible cysteine residues which can be reduced to free thiol groups for reaction to form the polymer conjugate. In preferred embodiments, the protein comprises albumin, which preferably is a serum albumin such as BSA. BSA can non-covalently bind on a surface (e.g., physical bonding), which grants the conjugate the ability to anchor on various surfaces, preferably without pretreatment.

In some embodiments, the proteins may be crosslinked after coating, resulting in a more stable and cohesive coating layer. A crosslinking agent such as glutaraldehyde can be employed for this purpose.

Once bound to the substrate surface, the polymer portion of the conjugate are exposed as “leaves”, providing functionality, examples of which will be described below. In some embodiments, the polymer may comprise a polyacrylate or a polymethacrylate.

In some embodiments, examples of functional polyacrylates or polymethacrylates include:
PSBMA (poly-sulfobetaine methacrylate)
PAMA: Poly-(2-Aminoethyl methacrylate hydrochloride)
PMTAC: Poly-[2-(Methacryloyloxy)ethyl]trimethylammonium chloride
PMPC: Poly-2-Methacryloyloxyethyl phosphorylcholine
PSPAK: Poly-3-Sulfopropyl methacrylate potassium salt
PHEMA: Poly-2-Hydroxyethyl methacrylate
PSA: Poly-Sodium acrylate
PDMA: Poly-dopamine methacrylamide

### Polyacrylates/Polymethacrylates Grafted on Native Bovine Serum Albumin (BSA) Protein Molecules.

In some embodiments, disclosed herein are BSA proteins combined with a polyacrylate comprising one of a broad selection of polyacrylates for desired functions. Such conjugates may be produced through the thiol-ene click reaction, for example.

In some examples, conjugated polyacrylates with desired functional groups can customize the physicochemical properties of protein coatings, imparting coating surfaces with versatile customized functionalities for biomedical applications. For example, the polyacrylate may comprise functional groups such as zwitterionic, charged, and/or stimuli-responsive groups.

In some embodiments, the polyacrylate imparts antifouling properties for implantable medical devices. In at least one example, the as-prepared antifouling coating comprises engineered proteins which are manufactured (e.g., produced or synthesized) by grafting sulfobetaine methacrylate (SBMA) segments on native bovine serum albumin (BSA) protein molecules. The SBMA part enables coating surfaces to exhibit superior resistance to biofouling for a broad spectrum of species (e.g., proteins, metabolites, cells, and biofluids) under various biological conditions. It is believed that the disclosed protein-based antifouling coating can resist over 99% bio-foulants from fetal bovine serums under complex biofluids conditions (e.g., various salinity and pH 3-10), and exhibits low-fouling properties and long-term stability in an in-vitro blood circulation test.

SBMA is a typical zwitterion with a more robust hydration layer than commercial PEG materials to resist biofouling. Moreover, zwitterions are generally more inert to pH and salinity changes in aqueous solutions than the charged functional groups, such as —COO⁻ and −NH₃⁺ groups, on native BSA protein surfaces.

The engineered design of the BSA@PSBMA protein integrates the natural function of native BSA, in surface-independent anchoring, on diverse target substrates with the artificially modified function of PSBMA, to prevent biofouling with strong interfacial hydration.

Unlike traditional synthetic polymers of which the coating operation requires arduous surface pretreatments, the engineered protein BSA@PSBMA can achieve facile and surface-independent coating on various substrates through a simple dipping/spraying method. In particular, the synthesized BSA@PSBMA material can be uniformly coated on various substrates, including metals, minerals, and plastics, through a one-step dipping or spraying method, preferably without the need of surface pretreatment.

Interfacial molecular force measurements and adsorption tests demonstrate that the substrate-foulant attraction is significantly suppressed due to strong interfacial hydration and steric repulsion of the structure of BSA@PSBMA, enabling coating surfaces to exhibit superior resistance to biofouling for a broad spectrum of species including proteins, metabolites, cells, and biofluids under various biological conditions.

In some embodiments, the polyacrylate provides stimuli-responsive groups such as pH-, light-, thermo-, or electro-responsive groups. Tunable or smart surfaces that exhibit stimuli-responsive functions can be used for targeted therapy, controllable drug release, and interventional imaging. which could be achieved via coupling stimuli-responsive polyacrylates with native proteins. For example, AMA (2-Aminoethyl methacrylate hydrochloride) with the pH-responsive amino groups (—NH₂) may be grafted on BSA to produce engineered protein BSA@PAMA.

In some embodiments, the polyacrylate provides robust adhesion with various micro- or nanomaterials, which can be used for therapeutic sensors. The adhesive coating serves as an interfacial binder can robustly immobilize various materials, including cellulose nanocrystals (CNC), cellulose fibers, microfibrillated cellulose, and silica in different forms such as silica spheres. Immobilizing nanomaterials on therapeutic sensors is commonly used in bioengineering applications, and therefore an interfacial binder that could ‘glue’ various nanomaterials comprises the engineered protein BSA@PMTAC, as it showed strong attraction to negatively charged surfaces.

### Synthesis

The synthesis of the polymer-protein conjugate comprising BSA and polyacrylates depends on the type of polyacrylate, reaction parameters, and concentration of BSA.

In at least one example, a zwitterion-conjugated protein is synthesized via grafting zwitterionic acrylate or methacrylate segments onto native BSA molecules through facile thiol-ene click chemistry. (Hoyle, Charles E., and Christopher N. Bowman. “Thiol-ene click chemistry.” Angewandte Chemie International Edition 49, no. 9 (2010): 1540-1573; and Tang, Wen, and Matthew L. Becker. ““Click” reactions: a versatile toolbox for the synthesis of peptide-conjugates.” Chemical Society Reviews 43, no. 20 (2014): 7013-7039, both of which are incorporated in their entirety herein by reference).

In alternative embodiments, the polymer can be synthesized first by click chemistry, atom transfer radical polymerization (ATRP), reversible addition-fragmentation chain transfer (RAFT) or another suitable polymerization method, and then grafted to the protein via click chemistry if the pre-synthesized polymer has a —SH thiol end. In another alternative, which is not preferred due to its requirement of catalysts, the sharpless asymmetric dihydroxylation may be suitable.

These alternative methods may not be preferred as they involve two steps and sometimes involve non-oxygen conditions. The thiol-ene click polymer grafting and polymerization under ambient conditions (room temperature in air) is more convenient and highly efficient.

### III. Example Synthesis Process

The following is an explanation of exemplary methods and processes for synthesizing BSA@Polymer, and BSA@PSBMA coatings.

### (i.) Example Process for Synthesizing BSA@Polymer.

FIG. 1 shows a schematic process of synthesizing BSA@Polymer via thiol-ene click chemistry.

Generally, a broad selection of functions for the protein-based coatings can be imparted through grafting polyacrylates with desired functional groups, on BSA molecules, via the radical-mediated thiol-ene click reaction under mild conditions, as shown in FIG. 1. The BSA molecule is first reduced to produce free thiol groups, which can then react with carbon-carbon double bonds in the acrylate or methacrylate monomers, followed by polymerization.

For example, synthesizing BSA@PSBMA based on BSA and sulfobetaine methacrylate (SBMA) can start with the reduction of disulfide bonds in BSA molecules (5 mg mL⁻¹) by NaBH₄ (200 mM) for 1 h (denote reduced BSA as re-BSA) to produce 1.42 mM free thiol groups (—SH) as detected using UV-vis spectrum (FIG. 2A) and X-ray photoelectron spectra (XPS) (FIG. 2B).

The more NaBH₄, the more free thiol groups in the re-BSA solution. Afterward, the re-BSA solution can be mixed with SBMA monomers (200 mM) to initiate the rapid click reaction between free thiol radicals and carbon-carbon double bonds in SBMA, during which most of the free thiol groups reacted with carbon-carbon double bonds within 1 h, as suggested in FIG. 2C. Finally, the polymerization may be maintained at ambient conditions (room temperature in air) for 30 h.

In this example, the resultant biopolymer was dialyzed and freeze-dried. Fourier transform infrared spectra (FTIR, FIG. 2D), XPS scan (FIG. 2E), and energy dispersive spectra (EDS, Figure S23) confirmed that the as-synthesized BSA@Polymer contained both the characteristic peak of polyacrylates and BSA, indicating the successful preparation of eight BSA@Polymers.

For example, the FTIR spectrum of BSA@PSBMA included amide peaks of BSA and —C═O and —SO₃ groups of SBMA monomer (FIG. 2D); XPS (FIG. 2E) and EDS double confirmed the presence of —SO₃ in engineered protein. FTIR spectra of BSA@PSPAK (SPAK, 3-Sulfopropyl methacrylate potassium) showed that the carbon-carbon double bond (980 and 1275 cm⁻²) in SPAK monomer was broken after polymerization and the presence of SPAK (—C═O group, 1726 cm⁻² and —SO₃, 1040 cm⁻²) and BSA (Amide I and Amide II) in the final product. The synthesized BSA@Polymers can easily be dip/spray coated on substrates to perform diverse functions for versatile scenarios.

### (ii.) Example Process for Synthesizing BSA@SMBA.

FIG. 8 shows an example process for synthesizing the BSA@PSBMA coating. As exemplified, the engineered protein BSA@PSBMA could be high-efficiently synthesized via a facile radical-mediated thiol-ene click reaction under mild conditions without side products.

In at least one example, disulfide bonds in BSA molecules (4 mg mL⁻¹, pH 7.5) are reduced to free thiol groups by NaBH₄ (50 mM) (denote reduced BSA as re-BSA). Free thiol group (—SH) was detected using the X-ray photoelectron spectra (XPS) of re-BSA in FIG. 9B and UV-vis spectrum after reduction.

Subsequently, the re-BSA solution was mixed with SBMA monomer (e.g., 400 mM, pH 7.5) to initiate the rapid click reaction between free thiol radicals and carbon-carbon double bonds of SBMA (FIG. 9A), and finally keeping the polymerization at ambient conditions for 30 h.

The resultant biopolymer was dialyzed and freeze-dried. The XPS characteristic peaks of BSA (O—C—NH—C) and SBMA (—N⁺ and —SO₃⁻) in FIG. 9B, and the thermogravimetric analysis of the resultant biopolymer indicated a successful conjugation of BSA and SBMA.

Fourier transform infrared (FTIR) (FIG. 9C) spectra of BSA@PSBMA further confirmed that the carbon-carbon double bond (1324 cm⁻²) in SBMA was broken after polymerization and the presence of SBMA (aldehyde group, —N⁺ and —SO₃) and BSA (Amide I and Amide II) in the final product.

An average of 1068 SBMA units was grafted on one BSA molecule as estimated from the FTIR spectra and energy dispersive spectroscopy.

To this end, the choice of thiol-ene click chemistry reaction enables both polymerization and conjugation of SBMA monomers on the BSA. The antifouling performance of BSA@PSBMA is due to bionic vine-thorn design and the polymer segment (PSBMA) inducing the defensive interfacial hydration layer, and additional steric repulsion between the coatings and foulants and gives the robust antifouling capability to this coating.

Further, it has been appreciated that the SBMA, without polymerization, may not provide the same antifouling features to the coating material. PSBMA has a longer chain length and exhibits anti-polyelectrolyte properties, where PSBMA will extend and resist the foulant approach surfaces in an environment with high salinity. The SBMA-g-SBMA, which has a monomer SBMA grafted on BSA, cannot maintain such antifouling performance because the SBMA monomer cannot exhibit anti-polyelectrolyte properties. Meanwhile, like seaweed, a shorter one could make the surface slippery but cannot be as effective as the longer one. Therefore, BSA@PSBMA coating overcomes the limitation of general protein/monomer-grafted-protein coating that can only work in mild conditions. The polymerization of SBMA plays the major role in antifouling property of this coating.

### IV. Test Results for BSA@Polymer

The following is a discussion of various tests and results of synthesizing the BSA@Polymer, as described above.

### (i.) Surface-Independent and Robust Anchoring of BSA.

The surface-independent, controllable, and robust anchoring of engineered proteins arises from the BSA-substrate interactions in the protein coating process.

In at least one example, the sticky BSA (5 mg mL⁻¹ dip-coating for 30 min) was coated on various substrates, including inorganics, organics, and metallics, with a typical thickness of ˜14.5 nm. After coating, the static water contact angle of substrates all changed from their initial contact angles (˜0° to ˜105°) to ˜60°.

In some examples, the prepared protein coatings were further stabilized via glutaraldehyde crosslinking (denoted as crosslinked BSA or cBSA), resulting in a slightly decreased contact angle (FIG. 3A).

Besides a universal anchoring property, BSA exhibited a controllable coating behavior, which was monitored in real-time using a quartz crystal microbalance with dissipation monitoring (QCM-D).

As shown in FIG. 3B, fast adsorption of BSA on the Au sensor was observed upon the introduction of BSA solutions into the QCM-D chamber. After stable adsorption was attained (˜1800 s), deionized water was introduced into the chamber to remove free and loosely bonded BSA. The QCM-D results indicated that a higher concentration of BSA in the solution would lead to faster protein adsorption and a thicker coating.

Solution pH also remarkably influenced the coating process (FIG. 3B). The adsorption rate was highest at pH near the isoelectric point (Ip, pH 4.8 for BSA), where the electrostatic repulsion among protein molecules was minimal.

Salinity also greatly altered the coating behavior of the proteins (FIG. 3B). It was found that 1 mM of multivalent salt ions could significantly increase the adsorption of proteins on substrates. For example, the BSA adsorption capability in an aqueous solution with Cu²⁺ ions was 4 times higher than that in the pure BSA solution, as displayed in FIG. 3B. The salt ions could neutralize the surface charges of proteins to decrease the electrostatic repulsion or change the protein secondary structures to enhance the attraction between substrates and protein molecules, accelerating the protein coating process.

After being coated on substrates, the proteins could robustly anchor on the substrates even in harsh conditions, including ultrasound, varying pH, surfactant attack, and enzymatic digestion treatments. The atomic force microscope (AFM) and laser scanning confocal microscope (LSCM) images in FIG. 3C displayed the BSA proteins were uniformly coated on silicon surfaces. Although some proteins detached from the substrates during the harsh environment treatments, the coating coverage remained higher than 85%.

After crosslinking with glutaraldehyde, the cBSA coatings could stably anchor on substrates and maintain similar surface coverage and wettability as that before treatments.

The crosslinked BSA proteins were more stable in harsh environments. The universal anchoring and robustness of protein coating most likely stemmed from the strong and adaptive interactions between BSA molecules and substrates. Anchoring strength of the coating layer can be tuned by solution chemistry, such as pH and salinity, resulting in a controllable coating process.

It is well-acknowledged that the interfacial molecular interactions between substrate surfaces and BSA proteins fundamentally govern the anchoring strength of coatings. Stronger interactions resulted in more robust anchoring. Therefore, the underlying anchoring mechanisms can be elucidated by directly measuring the interactions between BSA and target substrate surfaces.

The major non-covalent interactions in an aqueous solution include hydrogen bonding, hydrophobic force, electrostatic interactions, and other interactions (e.g., van der Waals force, cation-π, and anion-π interactions), as indicated in FIG. 4C. An interfacial binding usually hybridizes several different interactions under a specific condition, The interfacial interaction forces between BSA and substrates in aqueous solutions were measured using a surface forces apparatus (SFA) (FIG. 4D).

For all SFA experiments, 1 mM NaCl was used as a background aqueous solution and other salts were also introduced into the background solution to evaluate their impact on the interfacial molecular interactions.

In the SFA measurements, normalized force-distance (F/R-D) profiles and adhesion (anchoring forces) of BSA and to different model substrate surfaces were displayed in FIG. 4C. It was found that BSA-substrate interaction strengths were substrate-specific.

Specifically, BSA demonstrated comparable anchoring strength to both the hydrophilic mica surface (9.41 mN m⁻¹) and to the hydrophobic trichloro(octadecyl) silane (OTS) surface (7.02 mN m⁻¹) and higher adhesion to the metallic gold surface. The adhesion of BSA to mica increased with longer contact time as shown in FIG. 4D. The enhanced adhesion (7.93 mN m⁻¹) with time between BSA and hydrophilic mica could be mainly due to the strengthened hydrogen bonding. Hydrophobic interaction played an important role in the interactions of BSA to hydrophobic OTS surfaces.

FIG. 4E displayed that the adhesion of BSA to mica surfaces decreased with increasing the solution pH. Such decrease resulted from the increased electrostatic repulsion under the alkaline environment, agreeing with the lower adsorption of BSA shown in FIG. 3C. In the presence of 10 mM Ca²⁺ ions, the BSA-mica adhesion increased from 9.41 mN m⁻¹ to 12.97 mN m⁻¹ (FIG. 4F). Protein-substrate interactions can be complex in the presence of salt ions.

For example, Ca²⁺ ions could weaken hydrogen bonding between BSA and mica, as suggested by the increase in adhesion when the contact time increased from 1 min to 15 min, which was only 2.09 mN m⁻¹ (FIG. 4F), in contrast to the increased adhesion for the case without Ca²⁺ ions, which was 7.93 mN m⁻¹ in FIG. 4D. Ca²⁺ ions could also reduce electrostatic repulsion between BSA and mica via adsorbing on surfaces to neutralize the surface charge of both mica and BSA (Isothermal titration calorimetry measurement (ITC), FIG. 4G).

The reduced electrostatic repulsion compensated for the weakened hydrogen binding, leading to an increased adhesion between BSA and mica surfaces in the presence of Ca²⁺ ions. The other ions in the aqueous solutions also influenced the BSA-substrate adhesion, as displayed in FIG. 4H.

For example, the adhesion of BSA to the hydrophilic mica surface increased from 9.41 mN m⁻¹ to 13.75 mN m⁻¹ in the presence of 10 mM Fe³⁺ ions; and the adhesion forces to the hydrophobic OTS surface (43.8 mN m⁻¹) in 10 mM Fe³⁺ solution improved to 625% of the solution without Fe³⁺ ions (7.02 mN m⁻¹).

Generally, electrostatic attraction increased with the presence of ions (adhesion of contact for 1 min, FIG. 4H) due to the charge neutralization of protein surfaces, whereas the ions weakened the hydrogen binding at a different level. As a result, the adhesion forces at 15 min of contact were ion-specific, as indicated in FIG. 4H. The overall effects of multivalent ions on BSA adhesion are quite complex and might involve ion-protein affinity (following one of the Hofmeister Lyotropic series), ion-protein reactions, and ion-induced protein structure changes.

Nevertheless, the results of interfacial molecular force measurements demonstrated that the strong anchoring capability of BSA protein originated from multiple molecular interactions, and the strength of these interactions could adaptively change with the target surface properties and the chemistry of the surrounding aqueous solution, such as pH and salinity.

### (ii.) Applications of BSA@Polymer Coatings.

The diversity of engineered protein BSA@Polymer could impart substrate surfaces with tunable and versatile physicochemical properties for various applications, such as non-adhesion antifouling surface, pH-responsive coating for drug release, and adhesive interfacial glue, as indicated in FIG. 5A.

The interactions of BSA@PSBMA (blue) and BSA@PMTAC (red, MTAC, 2-methacryloyloxy ethyl trimethylammonium) to model hydrophilic surface were displayed in FIG. 5F, where BSA@PSBMA demonstrated near zero adhesion to model hydrophilic surfaces while BSA@PMTAC exhibited a strong adhesion. It is noted that, for the BSA@PSBMA, the BSA portion anchors on the substrate with strong adhesion; after anchoring, the PSBMA portion extends into the solution and covers the adhesive BSA layer. Therefore, the interaction of BSA@Polymer coatings arise from the outer polymer layer and surrounding substance, e.g., model hydrophilic surface-mica. There is almost no adhesion between PSBMA and mica, while a strong adhesion between PMTAC and mica.

The area between adhesion and non-adhesion was the transition domain with tunable interfacial interaction. BSA@PSBMA as an antifouling coating was demonstrated in this work. Specifically, gold substrates were first dip-coated in 5 mg mL⁻¹ BSA@PSBMA solution for 4 h. Then, the protein coating was patterned as indicated in FIG. 5B.

Finally, incubating the patterned protein-coated substrate in milk at 37° C. for 48 h before characterizing the foulant distribution by optical photothermal infrared (O-PTIR), which is a non-contact technique to precisely position foulants based on their characteristic infrared peaks. FIG. 5C clearly showed that non-coated parts of substrates were contaminated by the foulants from milk; while in BSA@PSBMA-coated areas, no foulant was founded.

The antifouling coatings also demonstrated excellent fouling resistance to other biological fluids, such as lipid (Canola oil) and fetal bovine serum, and the corresponding foulants distribution was displayed in FIG. 5D. The coverage of foulants on substrate surfaces was quantified by setting foulants coverage on bare substrate surfaces as 100% in FIG. 5E.

It was found that bare substrate surfaces were all covered with a large number of foulants, while BSA@PSBMA coatings resisted over 99% of foulants from all complex biofluids. This superior antifouling property of BSA@PSBMA most likely originated from its bionic structure with strong interfacial hydration and steric repulsion, hindering the initial attachment of foulants on surfaces.

Tunable or smart surfaces that exhibit stimuli-responsive functions are essential for targeted therapy, controllable drug release, and interventional imaging, which could be achieved via coupling stimuli-responsive polyacrylates with native proteins. For example, AMA (2-Aminoethyl methacrylate hydrochloride) with the pH-responsive amino groups (—NH₂) was grafted on BSA to produce engineered protein BSA@PAMA. The as-prepared BSA@PAMA coating exhibited pH-responsive behavior and was employed as a therapeutic carrier for controllable drug release, as indicated in FIG. 6A.

At pH 7, the amino groups in BSA@PAMA will be protonated, and therefore BSA@PAMA will be positively charged, leading to electrostatic attraction and loading the negatively charged model drug Congo Red (CR). At pH 9.5, the carboxyl groups on BSA will be deprotonated, and therefore BSA@PAMA will be negatively charged, leading to electrostatic repulsion and releasing the model drug CR (FIGS. 6A and 6B).

Based on this tunable performance, BSA@PAMA coated polyvinylidene fluoride (PVDF) membrane could reserve model drugs at pH 7 and then release them at pH 9.5, as indicated in FIG. 6C. The drug-releasing behavior of the BSA@PAMA coated PVDF and bare PVDF membrane was quantified using UV-Vis spectra (FIG. 6D). It was found that almost no drug was released from BSA@PAMA-coated PVDF at pH 7, while at pH 9.5, the drug was released from BSA@PAMA-coated surfaces rapidly.

The cumulative release of CR was displayed in FIGS. 6E and 6F, indicating that the release rate of CR from BSA@PAMA-coated PVDF membrane at pH 9.5 was 8.5 times higher than that at pH 7. BSA@PAMA coating is an example of stimuli-responsive coating for medical applications. Other BSA@Polymer coatings with tunable functions for customized applications could also be prepared based on the strategy in this work.

Immobilizing nanomaterials on therapeutic sensors is commonly needed for bioengineering applications, and therefore an interfacial binder that could ‘glue’ various nanomaterials has been developed based on the engineered protein BSA@PMTAC, as it showed strong attraction to the negatively charged surfaces (FIGS. 5A and 5F). 2-(methacryloyloxy)ethyltrimethylammonium chloride (MTAC) is a methacrylate with quaternary ammonium group that maintains positively charged to attract negatively charged nanomaterials, such as the rigid and hydrophilic cellulose nanocrystal (CNC), silica spheres, and cellulose nanofibers (FIG. 7A) on its surface.

FIG. 7A clearly showed that the CNC could not be immobilized on the substrate without the BSA@PMTAC coating. The reciprocating ball-on-disk friction test evaluated the stability of the immobilized CNC on BSA@PMTAC coating. The typical friction curves—friction force versus sliding distance under a certain normal loading force—were shown in FIG. 7B. The CNC/BSA@PMTAC coating was undergone over 1000 cycles of friction, in which the normal force increased from 5 mN to 40 mN in the initial stage and then decreased from 40 mN to 1 mN, then gradually increased back to 25 mN.

The friction coefficient between the ball and CNC/BSA@PMTAC coating under 10 mN of loading force was 0.46 during the 100-150 cycles of friction and remained almost unchanged after 950 cycles (FIG. 7C). It suggested that CNC/BSA@PMTAC coating maintained its structural integrity during the friction test, and CNC was firmly glued on the adhesive BSA@PMTAC surfaces.

These three applications are exemplary for introducing on-demand functions to engineered proteins, such as BSA@Polymer, and then applying these engineered proteins as coatings to enable versatile surface functions, such as adhesion and antifouling.

### V. Test Results of BSA@PSBMA

The following is a discussion of various tests and results of synthesizing the BSA@PSMBA, as described above.

### (i.) Physical Properties of BSA@PSBMA

The zeta potential results in FIG. 9D showed that the isoelectric point (Ip) of BSA@PSBMA was at pH 3.3, lower than that of native BSA at pH 4.8, due to the conjugation of PSBMA.

To test its anchoring capability, BSA@PSBMA was deposited on metallic, inorganic, and organic substrates by either spraying or dipping methods (FIG. 9E). The lower static water contact angles on BSA@PSBMA-coated surfaces than on native BSA-coated surfaces (FIG. 9E) resulted from the higher hydrophilicity of BSA@PSBMA. The coating thickness was regulated by tuning solution pH, reactant concentrations, and coating time.

The thickness of BSA@ PSBMA coating on mica surfaces under different pH conditions was measured using a surface forces apparatus. FIG. 9F shows that the thickness of BSA@PSBMA coating at pH 3 steadily rose to ≈36 nm in 4 h, and further prolonging the coating time would not significantly increase the coating thickness.

At pH 5, the thickness was only about 18 nm after 4 h. Such different coating behaviors most likely stem from the increased adsorption rate of proteins at pH near Ip, where the electrostatic repulsion among molecules is minimal.

With a longer coating time, the coverage of proteins on surfaces could increase and surface roughness would decrease, benefiting the antifouling performance. The successful synthesis and facile preparation of BSA@PSBMA coatings enable the following antifouling performance tests at micro and macro scales.

### (ii.) Anti-fouling Performance of BSA@PSBMA-Coated Surfaces.

Fouling is a spontaneous process in biosystems associated with the dynamic adsorption and accumulation of biomolecules on surfaces, which could be directly monitored in real-time using a quartz crystal microbalance with dissipation monitoring (QCM-D) (FIG. 10A).

The antifouling performance of BSA@PSBMA-coated surfaces against various model foulants from biosystems was screened using a QCM-D, where the bare Au sensor and native BSA-coated Au sensor were used as the control group.

As shown in FIG. 10B, a strong negative frequency shift was observed for all test sensors upon the introduction of milk into the QCM-D chamber, indicating the adsorption of foulants from milk on all tested surfaces.

After stable frequency was attained, ultrapure water was introduced into the chamber to remove the loosely bonded foulants on sensors, giving rise to an increase in frequency. This milk-water alternative rinsing was continuously conducted six times. The almost complete reversible adsorption-desorption of milk on the BSA@PSBMA-coated sensor demonstrated excellent antifouling performance of BSA@PSBMA coatings.

BSA@ PSBMA-coated sensors also exhibited high-efficiency antifouling capability to a broad spectrum of biofoulants, including biofluids (e.g., bovine serum, egg white, and yolk), common proteins (concanavalin A (Con A), collagenase, BSA, lysozyme (Lyso), mucin from porcine stomach), carbohydrate in body fluids (sucrose, fructose, humic acid), and small biological signaling molecules like dopamine (FIG. 10C). For instance, the remnant bovine serum on the BSA@PSBMA-coated sensor after rinsing with water was ˜50.3 ng cm 2, while those on the native BSA-coated sensor and bare Au sensor were as high as ˜1131 ng cm-2 and ˜2313 ng cm 2, respectively.

### (iii.) Long-Term Operation (Macro-Scale Tests).

Long-term operation in macroscale bulk tests with real biological fluids (FIG. 11A) is of practical importance to evaluate the antifouling capability of coatings, in addition to short-period dynamic adsorption of biomacromolecules at the nanoscale by QCM-D in biological buffers.

In macroscale tests, the tested surfaces would undergo more complicated chemical and biological processes, such as the formation of biofilms and the colonization of microorganisms on surfaces, which primarily cause irreversible dysfunction of devices and healthcare-associated infection. In these tests, bare substrate surfaces and surfaces coated with native BSA and BSA@PSBMA were incubated in representative biofluids at 37° C. for 48 h (FIG. 11A), including milk, lipid (Canola oil), Chinese hamster ovary (CHO) cells nutrient mixture, and fetal bovine serum (FBS).

All tested surfaces were rinsed with ultrapure water before the characterizations by optical photothermal infrared (O-PTIR) spectroscopy, which is a noncontact technique that couples a visible laser with an infrared laser to precisely position foulants based on their characteristic infrared (IR) peaks, as shown in FIG. 11B.

Biofoulants on the surfaces incubated in milk were mapped in FIG. 11C, based on their characteristic IR peak at 1650 cm−1 (bottom spectra in FIG. 11B). Likewise, surfaces fouled in the lipids, CHO cells, and bovine serum were also characterized in FIG. 11C according to their individual characteristic IR peaks.

FIG. 4D indicated that a large number of foulants adhered on all bare substrate surfaces, less on native BSA-coated surfaces except for the lipid, and nearly zero fouling on BSA@PSBMAcoated surfaces in all biofluids.

The coverage of biofoulants on surfaces was quantified in FIG. 4C by setting biofoulants coverage on bare substrate surfaces as 100%. The results in bulk fouling tests are consistent with the nanoscale adsorption tests, both demonstrating remarkably reduced biofouling on BSA@PSBMA-coated surfaces. This superior antifouling property most likely originated from the bionic hierarchical structure of BSA@PSBMA that provides strong interfacial hydration and steric repulsion to hinder the initial attachment of foulants on surfaces.

(iv.) Complex Biological Fluids with Diverse pH and Salinity Conditions.

The antifouling capability of BSA@PSBMA coatings was further examined in more complex biological fluids with diverse pH and salinity conditions which could greatly compromise the antifouling performance in practice. Such complex biological conditions inevitably occur after the medical device implantation or during clinic testing, which leads the surrounding environment of the antifouling coatings extremely complex.

In the following bulk tests, protein-coated surfaces were incubated in fetal bovine serum (FBS) under various pH and salinity conditions. Specifically, bare Au surfaces and Au surfaces coated with native BSA and BSA@PSBMA were incubated in FBS at pH 3.6, 6.8, 7.4, and 9.8 or with various salinity (250 mM for KCl and NaCl, 10 mM for CaCl₂) and MgCl₂) for 48 h at 37° C.

The coverage of biofoulants on different surfaces is summarized in FIGS. 12A and 12B, and the corresponding distribution of foulants on surfaces was displayed in FIGS. 12C and 12D.

It was found that BSA@PSBMA-coated surfaces exhibited high-efficient resistance to over 99.9% of the adsorption of biomacromolecules in FBS under high salinity and various pH conditions, over tenfold superior to native BSA-coated surfaces in antifouling capability.

Such extraordinary antifouling performance of BSA@PSBMA could be attributed to its bionic “vine-thorn” design.

In particular, the hydrophilic SBMA part endowed the coating with a strong interfacial hydration layer and additional steric repulsions that greatly hindered the initial attachment of foulants on surfaces, while the bare BSA coating failed to resist biofouling under complex biological conditions. The less biofouling on Au surfaces at higher pH conditions (FIG. 12A) could be attributed to the lower settling rate of foulants due to the higher electrostatic repulsion between foulants and substrates. High salinity resulted in worse biofouling on surfaces, as suggested in FIGS. 12B and 12D. The same trend was also observed in the dynamic adsorption of FBS (FIG. 12E) on protein-coated surfaces.

The adsorption of FBS at different pH and salinity (FIGS. 12F and 12G) clearly demonstrated the superior antifouling performance of BSA@PSBMA coating.

For instance, at pH 7.4, foulants adsorbed on the native BSA-coated surface were 925 ng cm⁻², while on the BSA@PSBMA-coated surface were as low as 6.24 ng cm⁻². For FBS with 250 mM KCl, the adsorption on the BSA-coated surface was 2445 ng cm⁻², whereas that on the BSA@PSBMA-coated surface was only 146.5 ng cm⁻².

With higher salt concentration (FIG. 12G), the adsorption amount of biofoulants decreased on both native BSA and BSA@PSBMA coatings, which was fundamentally determined by the comprehensive intermolecular interactions between foulants and the coating surfaces.

Specifically, the increased ionic strength at high salinity could suppress the electrostatic repulsion between negatively charged foulants and BSA molecules at coating surfaces, increasing attraction between foulants and surfaces.

In contrast, the high concentration of salt ions might weaken attractive intermolecular interactions like the hydrogen bonding between foulants and BSA proteins, decreasing attraction between foulants and surfaces.

The interfacial interaction forces between the protein-coated surfaces and foulants from biofluids will be investigated by direct interaction force measurements.

### (v.) Intermolecular Interactions

It is well-acknowledged that the biofouling process in different lengths and time scales is fundamentally governed by intermolecular interactions between substrate surfaces and foulants. Attractive interactions would lead to fouling, while repulsive ones result in nonfouling, as indicated in FIG. 13A.

Therefore, direct measurement of the intermolecular interactions between protein-based coating and foulants is critical in elucidating the underlying antifouling mechanisms and advancing the development of novel antifouling materials.

In at least one example, a mica surface was modeled as a hydrophilic foulant substrate, and trichloro(octadecyl) silane (OTS) composed of a long-chain alkyl group was set as the hydrophobic case.

The interfacial interactions between protein-coated surfaces and hydrophilic mica surface or hydrophobic OTS surface in aqueous solutions, at different contact time, were measured using an SFA (FIG. 13B).

In SFA measurements, normalized force-distance (F/R-D) profiles and adhesion between protein-coated surfaces and different model foulants are displayed in FIGS. 13C, 13D. It was found that adhesion (Fad/R) between BSA@PSBMAcoated surface and mica surface was only ≈0.95 mN m⁻¹ after 45 min of contact (FIG. 13C), fivefold lower than that between native BSA-coated surface and mica surface ≈4.11 mN m⁻¹.

Such low adhesion could be attributed to the steric repulsion of the “thorn” part on BSA@PSBMA that significantly counteracted attractive interfacial interactions, such as van der Waals (vdW) force, time-dependent hydrogen bonding, and other interactions (electrostatic force, cation-π interaction and ion bridging).

Likewise, the bionic structure induced steric repulsion granted zero adhesion between BSA@PSBMA coating and OTS, whereas the adhesion between native BSA coating and OTS reached up to ≈3.39 mN m⁻¹ (FIG. 13D).

Such weak interfacial attraction between the BSA@PSBMA coating and model foulants well accounted for the excellent resistance to biofouling in both nanoscale dynamic adsorptions (FIG. 10C) and macroscale bulk tests (FIG. 11D).

Interaction forces between protein-coated surfaces and model foulant surfaces in aqueous solutions with different pH and salinity were then systematically measured to investigate the underlying antifouling mechanisms of BSA@PSBMA in complex biological conditions.

FIGS. 13E, 13F present the interaction forces between a hydrophilic mica surface and proteincoated surfaces in aqueous solutions with various pH values.

It was found that the adhesion of the mica surface to the BSA@PSBMA-coated surface was as low as 0.45-0.98 mN m−1 in a wide range of pH values (FIG. 13E); however, the adhesion between native BSA-coated surface and mica surprisingly reached up to 8.06 mN m⁻¹ at pH 3.6 (FIG. 13F), which may be resulted from the strong electrostatic attraction under low pH conditions.

FIG. 13F also shows that the adhesion between the BSA and mica surfaces decreased with increasing the solution pH due to the increased electostatic repulsion under alkaline pH conditions.

FIG. 13G displayed the interaction forces of mica surfaces with protein-coated surfaces in a solution with the presence of 10 mM CaCl₂), and it clearly showed a zero adhesion and long-range steric repulsion between BSA@ PSBMA-coated surface and mica surface, whereas a dramatical high adhesion (≈12.71 mN m⁻¹) between native BSA-coated surface and mica surface.

Likewise, increased interfacial interactions between native BSA-coated surfaces and mica surfaces were found in all the tested solutions with the presence of salts (7-17.3 mN m−1, 10 mM of salts, and 15 min) as summarized in FIG. 13H, which were over tenfold higher than the adhesion between BSA@PSBMA-coated surfaces and mica surfaces FIG. 13I). Those results demonstrated very weak interfacial attraction between the BSA@PSBMA-coated surfaces and mica surfaces.

More importantly, such low interfacial attraction would not increase in the solutions with the presence of salts and in a wide range of pH values, implying the robust antifouling performance in practical complex biological conditions.

Such retained low interfacial interaction energy of engineered BSA@ PSBMA material arose from the hydrophilic zwitterionic PSBMA part on BSA, which exhibited a self-adapting antielectrolyte property and shifted from a compressed conformation to a relatively loose conformation in salt solutions, introducing long-range and strong steric repulsion to reduce the adhesion (FIG. 13G). Furthermore, the association constant, Ka, between native BSA molecule and Ca²⁺ ions was as high as 1.02×10⁶ in isothermal titration assays.

As a result, a large amount of Ca²⁺ ions were adsorbed on the native BSA coating surface from the surrounding environments, decreasing its surface charge density and weakening its binding with interfacial water molecules, causing dehydration on the surface of native BSA coating, as indicated in FIG. 13A.

The lower surface charge density reduced the electrostatic repulsion and dehydration lowered repulsive steric hydration force, resulting in significantly increased adhesion between native BSA-coated surfaces and foulants surfaces.

Fortunately, the engineered BSA@PSBMA was able effectively address such salinity-induced unstable interfacial hydration issue because of the low Ka of Ca²⁺ with BSA@PSBMA (≈160.3), rendering an unfailing low interfacial interaction and less fouling under complex biological conditions.

The antifouling properties of native protein coatings could be weakened with the presence of different ions due to multiple interactions between the ions and proteins, including the protein affinity with ions (generally following the Hofmeister Lyotropic series, where Ca²⁺ ions demonstrated higher affinity to proteins that Mg²⁺ ions.), ionic valence, ion-condensation, and ion-correlation interactions.

Nevertheless, our bionic design of BSA@PSBMA demonstrated its unfailing low interfacial interaction energy with other surfaces and high-efficient fouling resistance to biomacromolecules in complex biological fluids/buffers, paving the way for practical applications.

### (vi.) Example Application Testing.

The BSA@PSBMA coating with excellent antifouling properties holds great potential to be used in biomedical applications, such as on-skin biosensors, implanted microsensors, and artificial substitutes, as indicated in FIG. 14A. One of the most commonly used artificial substitutes is the artificial blood vessels that replace arterial vessels defunctionalized by the fouling of atherosclerotic plaque in coronary heart disease.

Such undesirable biofouling should be absolutely avoided after the implantation of new artificial vessels because 5 ng cm−2 of fibrinogen adsorption can cause blood coagulation on the newly implanted artificial blood vessel surfaces.

Here, it was demonstrated that a BSA@PSBMA-coated low-fouling artificial blood vessel, allowing stable operation in a long-term in vitro blood circulation test (FIG. 14B). After operation for 16 days, the flowing rate of blood in the BSA@PSBMA-coated medical tube was 8.98 cm s−1, which was 92.0% of the original flowing rate.

That of the native BSA-coated tube was 7.09 cm s⁻¹, 73.3% of the initial rate, and for the pristine tube, it rapidly dropped down to 4.37 cm s⁻¹, 49.11% of the initial (FIG. 14C). The decrease in blood flowing rate resulted from the in-stent restenosis in which the inner tube narrowed down with the accumulation of biofoulants on the inner surfaces. FIG. 14D displayed the surface morphology of the inner wall of the medical tubes after operation for 16 days.

It can be seen that large foulant debris fully covered the inner surface of the pristine tube, smaller and sporadic foulants on the native BSA-coated inner surface, and no obvious biofouling on the BSA@PSBMA-coated inner surface.

As a result, more carbon and oxygen, the main composition of biofoulants, were found on inner surfaces as displayed in EDS mapping (FIG. 14F) and linescan (FIG. 14G) of C+O elements. Besides, the fouling release property of the BSA@PSBMA-coated surface was demonstrated in a self-cleaning test (FIG. 14H).

In the test, the “UA” pattern prepared in by spraying method was first contaminated by viscous oil, then immersed in pure water, and finally, oil detached from the BSA@PSBMA-coated surface within 13 s. Such fouling release property of BSA@PSBMA coating was further supported by the induction timer test, in which a viscous oil droplet was brought to the surface and then retraced from the surfaces without any oil residual left behind on the BSA@PSBMA-coated surface.

The combination of excellent resistance to foulants attachment and fouling release performance in an aqueous environment implied the potential of the engineered BSA@PSBMA protein material in clinical utility.

### VI. Interpretation

Various systems or methods have been described to provide an example of an embodiment of the claimed subject matter. No embodiment described limits any claimed subject matter and any claimed subject matter may cover methods or systems that differ from those described below. The claimed subject matter is not limited to systems or methods having all of the features of any one system or method described below or to features common to multiple or all of the apparatuses or methods described below. It is possible that a system or method described is not an embodiment that is recited in any claimed subject matter. Any subject matter disclosed in a system or method described that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling may be used to indicate that an element or device can electrically, optically, or wirelessly send data to another element or device as well as receive data from another element or device. As used herein, two or more components are said to be “coupled”, or “connected” where the parts are joined or operate together either directly or indirectly (i.e., through one or more intermediate components), so long as a link occurs. As used herein and in the claims, two or more parts are said to be “directly coupled”, or “directly connected”, where the parts are joined or operate together without intervening intermediate components.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

The present invention has been described here by way of example only, while numerous specific details are set forth herein in order to provide a thorough understanding of the exemplary embodiments described herein. However, it will be understood by those of ordinary skill in the art that these embodiments may, in some cases, be practiced without these specific details. In other instances, well-known methods, procedures and components have not been described in detail so as not to obscure the description of the embodiments. Various modification and variations may be made to these exemplary embodiments without departing from the spirit and scope of the invention, which is limited only by the appended claims.

1. A protein-polymer conjugate comprising an acrylate or methacrylate polymer conjugated to a globular protein molecule, such as a serum albumin, preferably bovine serum albumin.

2. The conjugate of claim 1, wherein the polymer comprises a methacrylate polymer comprising zwitterionic methacrylate polymer.

3. The conjugate of claim 2 wherein the zwitterionic methacrylate polymer comprises poly sulfobetaine methacrylate (PSMBA).

4. The conjugate of claim 1 wherein the polymer comprises a methacrylate polymer comprising pH-responsive amino groups.

5. The conjugate of claim 4 wherein the methacrylate polymer comprises poly 2-Aminoethyl methacrylate (PAMA).

6. The conjugate of claim 1 wherein the polymer comprises a methacrylate polymer comprising a quaternary ammonium group methacrylate polymer.

7. The conjugate of claim 6 wherein the methacrylate polymer comprises one of poly 2-(methacryloyloxy)ethyltrimethylammonium chloride (PMTAC), poly 2-Methacryloyloxyethyl phosphorylcholine (PMPC), poly 3-Sulfopropyl methacrylate potassium salt (PSPAK), poly 2-Hydroxyethyl methacrylate (PHEMA), or poly dopamine methacrylamide (PDMA).

8. The conjugate of claim 1 wherein the polymer comprises an acrylate polymer comprising poly sodium acrylate (PSA).

9. The conjugate of claim 1 wherein the serum albumin is bovine serum albumin.

10. An anti-fouling coating on a substrate, comprising a layer of a conjugate of claim 1 non-covalently bonded to the substrate.

11. The coating of claim 10, wherein the coating is produced by spraying or dipcoating an aqueous solution of the conjugate, such that the conjugate binds non-covalently with the substrate.

12. The coating of claim 11 wherein the aqueous solution has a salt concentration and/or a pH level which has been varied to increase the binding of the conjugate to the substrate.

13. A method of conjugating acrylate or methacrylate monomers having carbon-carbon double bonds to proteins to produce a protein-polymer conjugate, comprising the steps of:
reducing the proteins to produce free thiol groups;
contacting the reduced proteins with the methacrylate or acrylate monomers to react the carbon-carbon double bonds to the free thiol groups; and
polymerizing the methacrylate or acrylate monomers.

14. The method of claim 12 wherein the monomers comprises zwitterionic methacrylate monomers.

15. The method of claim 13 wherein the zwitterionic methacrylate monomers comprise sulfobetaine methacrylate (SMBA).

16. The method of claim 12 wherein the monomers comprise methacrylates comprising pH-responsive amino groups.

17. The method of claim 15 wherein the monomers comprise 2-Aminoethyl methacrylates (PAMA).

18. The method of claim 12 wherein the monomers comprise quaternary ammonium group methacrylates.

19. The method of claim 17 wherein the monomers comprise comprise one of 2-(methacryloyloxy)ethyltrimethylammonium chloride (MTAC), 2-Methacryloyloxyethyl phosphorylcholine (MPC), 3-Sulfopropyl methacrylate potassium salt (SPAK), 2-Hydroxyethyl methacrylate (HEMA), or dopamine methacrylamide (DMA).

20. The method of claim 12 wherein the monomers comprise sodium acrylates (SA).
